(19) United States
(12) Patent Application Publication
(10) Pub. No.: US 2026/0069588 A1
(43) Pub. Date: Mar. 12, 2026

(54) VEPDEGESTRANT FOR USE IN TREATING CANCER

(71) Applicant: Arvinas Operation, Inc., New Haven, CT (US)

(72) Inventors: Ronald Peck, Cheshire, CT (US); lan Charles Anthony Taylor, Madison, CT (US); Chi Fung Keung, Quincy, MA (US); Sibyl Anderson, New York, NY (US); Sheryl Maxine Gough, Stratford, CT (US); John J. Flanagan, Guilford, CT (US)

(21) Appl. No.: 19/107,535

(22) Filed: Feb. 27, 2025

Related U.S. Application Data
(60) Provisional application No. 63/402,671, filed on Aug. 31, 2022.

(51) Int. Cl. A61K 31/496; A61P 35/00; A61P 35/04

(52) U.S. Cl. CPC A61K 31/496; A61P 35/00; A61P 35/04

(57) ABSTRACT

Disclosed herein are methods for treating cancer comprising administering to a subject a daily dose of Compound A, or a pharmaceutically acceptable salt thereof, as a single agent.

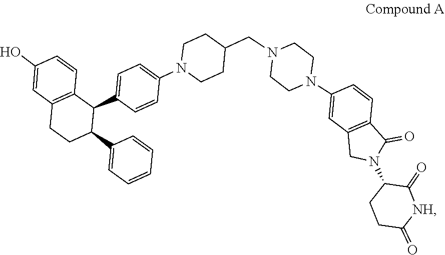 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

### CROSS-REFERENCES TO RELATED APPLICATIONS

This application claims priority to, and the benefit of, U.S. Application No. 63/445,950, filed Feb. 15, 2023, U.S. Application No. 63/431,082, filed Dec. 8, 2022, and U.S. Application No. 63/402,671, filed Aug. 31, 2022, the entirety of each of which is incorporated by reference herein.

### BACKGROUND OF THE INVENTION

Certain bifunctional compounds can target specific cellular proteins for degradation via the ubiquitin-proteasome system. Examples of such proteolysis targeting chimeric compounds (i.e., “PROTAC® protein degraders”) that target the Estrogen Receptor (ER) for ubiquitination and subsequent degradation are disclosed in International Publication No. WO 2018/102725, which is incorporated herein by reference in its entirety. Such bifunctional molecules exhibit a range of pharmacological activities consistent with the degradation of the ER including, but not limited to, treatment or amelioration of a disease condition such as cancer (e.g., breast cancer, uterine cancer, ovarian cancer, prostate cancer, endometrial cancer), or endometriosis.

A bifunctional molecule of particular interest is vepdegestrant (i.e., (S)-3-(5-(4-((1-(4-((1R,2S)-6-hydroxy-2-phenyl-1,2,3,4-tetrahydronaphthalen-1-yl)phenyl)piperidin-4-yl)methyl)piperazin-1-yl)-1-oxoisoindolin-2-yl)piperidine-2,6-dione or (3S)-3-[1,3-dihydro-1-oxo-5-[4-[[1-[4-[(1R,2S)-1,2,3,4-tetrahydro-6-hydroxy-2-phenyl-1-naphthalenylphenyl]-4-piperidinyl]methyl]-1-piperazinyl]-2H-isoindol-2-yl]-2,6-piperidinedione (referred to herein as “Compound A” or “Cpd A”)), which has the molecular formula of C₄₅H₄₉N₅O₄ and the following structure:

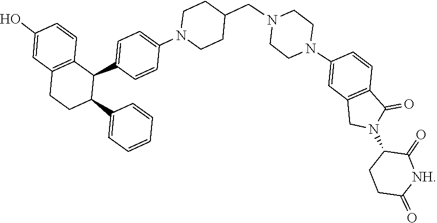 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

Compound A is under development as a PROTAC® protein degrader that targets estrogen receptor (ER) for the potential treatment of breast cancer and has been shown to be a useful modulator of targeted protein ubiquitination and degradation via the ubiquitin-proteasome pathway.

There is a need for appropriate dosage regimens of Compound A as an oral agent for treating cancers (e.g., breast cancer), to improve its benefit, including safety and efficacy, and convenience to patients while at the same time minimizing adverse events and risks to patients.

### SUMMARY OF THE INVENTION

The present invention provides, in part, dosage regimens for administering Compound A, or a pharmaceutically acceptable salt thereof, to a subject as a single agent for treating cancer. This summary is provided to introduce a selection of concepts in a simplified form that are further described below in the detailed description. This summary is not intended to identify key features or essential features of the claimed subject matter, nor is it intended to be used in isolation as an aid in determining the scope of the claimed subject matter.

Provided herein is a method for treating cancer comprising administering to a subject a daily dose of Compound A:

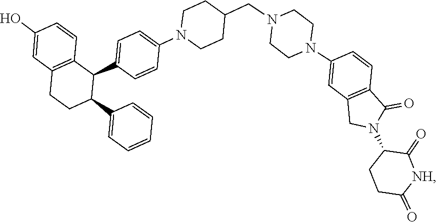 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

or a pharmaceutically acceptable salt thereof, wherein the daily dose of Compound A is about 100 mg or about 200 mg.

Provided herein is a method for treating cancer comprising administering to a subject a daily dose of Compound A:

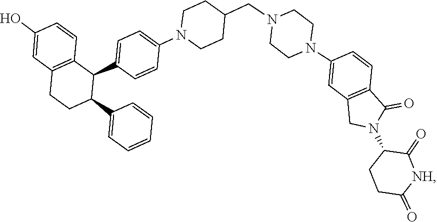 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

wherein the daily dose of Compound A is about 100 mg or about 200 mg.

In embodiments, the daily dose of Compound A, or the pharmaceutically acceptable salt thereof, is administered once per day (QD).

In embodiments, the daily dose of Compound A, or the pharmaceutically acceptable salt thereof, is administered orally to the subject.

In embodiments, the subject is in a fed state.

In embodiments, the cancer is breast cancer, lung cancer, colon cancer, brain cancer, head and neck cancer, prostate cancer, stomach cancer, pancreatic cancer, ovarian cancer, melanoma, endocrine cancer, uterine cancer, testicular cancer, or bladder cancer.

In embodiments, the cancer is breast cancer, lung cancer, prostate cancer, pancreatic cancer, or ovarian cancer.

In embodiments, the cancer is breast cancer, lung cancer, or prostate cancer.

In embodiments, the cancer is breast cancer. In embodiments, the breast cancer is metastatic or locally advanced. In embodiments, the breast cancer is estrogen receptor positive (ER+) breast cancer (e.g., human epidermal growth factor receptor 2 negative (HER2−)).

In embodiments, the subject is human.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated into and form a part of the specification, illustrate several embodiments of the present disclosure and, together with the description, serve to explain the principles of the disclosure. The drawings are only for the purpose of illustrating an embodiment of the disclosure and are not to be construed as limiting the disclosure. Further objects, features and advantages of the disclosure will become apparent from the following detailed description taken in conjunction with the accompanying figures showing illustrative embodiments of the disclosure, in which:

FIG. 1 shows the results of an exploratory analysis of ER expression in 14 paired biopsies from patients treated in various Part A dose escalation cohorts. The results indicated ER degradation (median decrease=67% [range: 21% to 89%]) at all doses of Compound A tested (up to 500 mg QD) and in tumors expressing either WT or mutant ER proteins.

FIG. 2A and FIG. 2B are the results of live-cell imaging used to measure proliferation of MCF7 cells (FIG. 2A) or T47D cells (FIG. 2B) for up to 6 days with various concentrations of Compound A or fulvestrant (15 pM-100 nM). Full growth of the cells over this time period is shown by extent of cell confluence with dimethyl sulfoxide (DMSO). In MCF7 cells, Compound A and fulvestrant had half-maximal growth inhibition (GI₅₀) values of 4 nM and 0.4 nM, respectively. In T47D cells, Compound A and fulvestrant had GI₅₀ values of 1.7 and 0.8 nM, respectively.

FIG. 3A and FIG. 3B show the results of tumor growth inhibition experiments (mean tumor volume (mm³) vs. time) associated with oral, once daily administration of Compound A at doses of 3 mg/kg, 10 mg/kg, and 30 mg/kg for 28 days compared to vehicle. At doses of 3 mg/kg, 10 mg/kg, and 30 mg/kg of Compound A, tumor growth inhibition (TGI) of 85%, 98%, and 124%, respectively, was observed compared to a control group in a MCF7/estradiol xenograft model (FIG. 3A). The 30 mg/kg dose was associated with a mean AUC₀₋₂₄ of 5,717 ng·h/mL. The tumor ER levels were reduced by ≥94% at all doses compared to mice administered vehicle only (FIG. 3B).

FIG. 4 shows the results of tumor growth inhibition (mean tumor volume (mm³) vs. time) experiments in female severe combined immunodeficient mouse in non-obese background (NOD/scid) mice harboring patient-derived xenograft model expressing ER (Y537S) a ESR1 (Y537S). Mice were administered of Compound A at an oral, once daily dose of 10 mg/kg or 30 mg/kg for 27 days, or fulvestrant (200 mg/kg, subcutaneous twice/week, for 2 weeks, followed by once-weekly for 2 weeks) for 27 days. Tumor growth was evaluated twice weekly by measuring tumor volumes until study termination. At study termination, Compound A (10 mg/kg), Compound A (30 mg/kg), and fulvestrant inhibited tumor growth by 99%, 106%, and 62%, respectively.

### DETAILED DESCRIPTION OF THE INVENTION

The present invention may be understood more readily by reference to the following detailed description of the embodiments of the invention and the Examples included herein. It is to be also understood that the terminology used herein is for the purpose of describing specific embodiments only and is not intended to be limiting. Compound A:

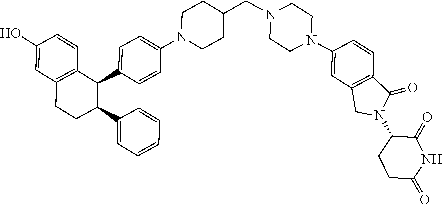 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

is under development as a PROTAC® protein degrader that targets estrogen receptor (ER) for the potential treatment of breast cancer and has been shown to be a useful modulator of targeted protein ubiquitination and degradation via the ubiquitin-proteasome pathway.

Compound A and pharmaceutically acceptable salts thereof are disclosed in International Publication No. WO 2018/102725 and U.S. Pat. Nos. 10,647,698, 10,899,742 and 11,104,666; International Publication No. WO 2021/041348; U.S. Ser. No. 17/472,847; U.S. Ser. No. 17/548,842; and U.S. Ser. No. 17/873,748. The contents of each of the foregoing references are incorporated herein by reference in their entirety.

### Definitions

Unless otherwise defined herein, scientific, and technical terms used in connection with the present invention have the meanings that are commonly understood by those of ordinary skill in the art.

The invention described herein suitably may be practiced in the absence of any element(s) not specifically disclosed herein.

As used herein, the singular form “a,” “an,” and “the” include plural references unless indicated otherwise. For example, “a” substituent includes one or more substituents.

As used herein, the term “about” when used to modify a numerically defined parameter (e.g., the dose of Compound A) means that the parameter may vary by as much as 10% below or above the stated numerical value for that parameter. For example, a dose of about 5 mg means 5 mg 10%, i.e., it may vary from 4.5 mg to 5.5 mg.

As used herein, terms, including, but not limited to, “agent,” “composition,” “compound,” “drug,” and “therapeutic agent” may be used interchangeably to refer to compounds included in the methods and uses of the present disclosure.

As used herein, the terms, “subject,” “participant,” and “patient,” are used interchangeably, to refer to any animal, including mammals. Mammals according to the disclosure include canine, feline, bovine, caprine, equine, ovine, porcine, rodents, lagomorphs, primates, humans, and the like, and encompass mammals in utero. In embodiments, humans are suitable subjects. Human subjects may be of any gender and at any stage of development.

Compound A is a Biopharmaceutics Classification System Class IV compound (low solubility/low permeability). Compound A can interconvert to its epimer, Compound B:

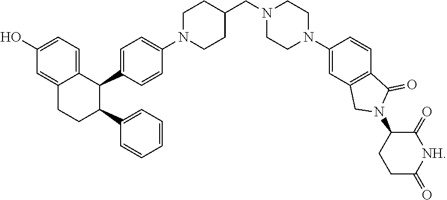 [SMILES: O=C1CC[C@@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

Preclinical data demonstrates that the exposure of Compound B is limited compared to Compound A (<26%). Compound B does not degrade the ER; however, Compound B shows similar antagonism of ER-dependent transcription compared to Compound A.

Other embodiments relate to the pharmaceutically acceptable salts of Compound A. Pharmaceutically acceptable salts of Compound A include the acid addition and base addition salts thereof.

Other embodiments also relate to the pharmaceutically acceptable acid addition salts of Compound A. Suitable acid addition salts are formed from acids which form non-toxic salts. Non-limiting examples of suitable acid addition salts, i.e., salts containing pharmacologically acceptable anions, include, but are not limited to, the acetate, acid citrate, adipate, aspartate, benzoate, besylate, bicarbonate/carbonate, bisulphate/sulphate, bitartrate, borate, camsylate, citrate, cyclamate, edisylate, esylate, ethanesulfonate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methanesulfonate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, p-toluenesulfonate, tosylate, trifluoroacetate and xinofoate salts.

Additional embodiments relate to base addition salts of Compound A. Suitable base addition salts are formed from bases which form non-toxic salts. Non-limiting examples of suitable base salts include the aluminum, arginine, benzathine, calcium, choline, diethylamine, diolamine, glycine, lysine, magnesium, meglumine, olamine, potassium, sodium, tromethamine, and zinc salts.

Compound A can form a wide variety of salts with various inorganic and organic acids. The acids that may be used to prepare pharmaceutically acceptable acid addition salts of Compound A are those that form non-toxic acid addition salts, e.g., salts containing pharmacologically acceptable anions, such as the hydrochloride, hydrobromide, hydroiodide, nitrate, sulfate, bisulfate, phosphate, acid phosphate, isonicotinate, acetate, lactate, salicylate, citrate, acid citrate, tartrate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate and pamoate [i.e., 1,1′-methylene-bis-(2-hydroxy-3-naphthoate)] salts. Compound A may form pharmaceutically acceptable salts with various amino acids, in addition to the acids mentioned above.

The chemical bases that may be used as reagents to prepare pharmaceutically acceptable base salts of Compound A are those that form non-toxic base salts with Compound A. Such non-toxic base salts include, but are not limited to, those derived from such pharmacologically acceptable cations such as alkali metal cations (e.g., potassium and sodium) and alkaline earth metal cations (e.g., calcium and magnesium), ammonium or water-soluble amine addition salts such as N-methylglucamine-(meglumine), and the lower alkanolammonium and other base salts of pharmaceutically acceptable organic amines.

Hemisalts of acids and bases may also be formed, for example, hemisulphate, and hemicalcium salts.

For a review on suitable salts, see Handbook of Pharmaceutical Salts: Properties, Selection, and Use by Stahl and Wermuth (Wiley-VCH, 2002). Methods for making pharmaceutically acceptable salts of Compound A are known to one of skill in the art.

### Dosage Regimens

Provided herein are methods for treating cancer comprising administering to a subject a daily dose of Compound A:

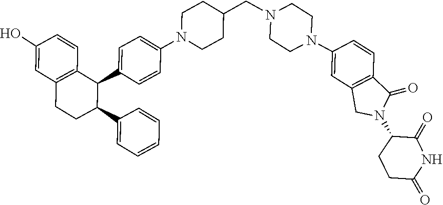 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

pharmaceutically acceptable salt thereof.

In embodiments, the daily dose of Compound A:

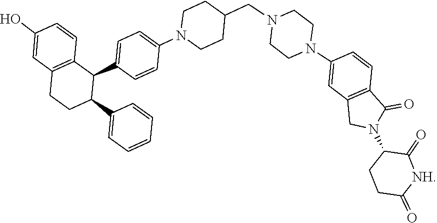 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

or a pharmaceutically acceptable salt thereof, is administered once per day (QD).

In embodiments, the daily dose of Compound A:

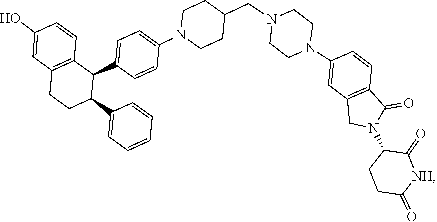 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

or a pharmaceutically acceptable salt thereof, is administered orally to the subject.

In embodiments, the subject is in a fed state.

In embodiments, the daily dose of Compound A:

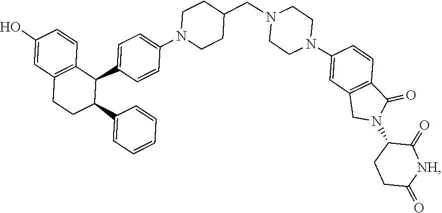 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

or a pharmaceutically acceptable salt thereof, is about 100 mg or about 200 mg, or an equivalent amount of a pharmaceutically acceptable salt thereof.

In embodiments, the daily dose of Compound A:

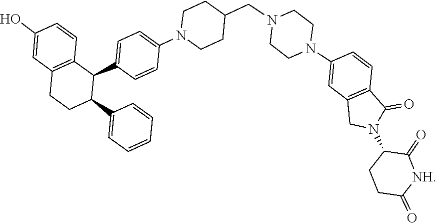 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

or a pharmaceutically acceptable salt thereof, is about 100 mg, or an equivalent amount of a pharmaceutically acceptable salt thereof.

In embodiments, the daily dose of Compound A:

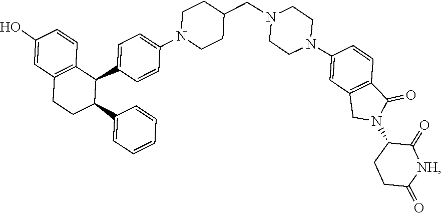 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

or a pharmaceutically acceptable salt thereof, is about 200 mg, or an equivalent amount of a pharmaceutically acceptable salt thereof.

In embodiments, the daily dose of Compound A:

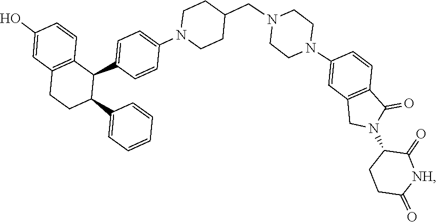 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

is about 200 mg.

In embodiments, the daily dose of Compound A:

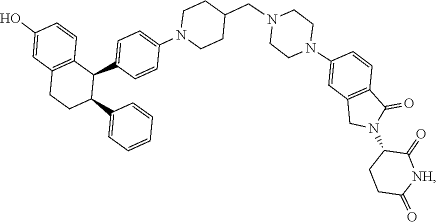 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

is about 100 mg.

In embodiments, the daily dose of Compound A:

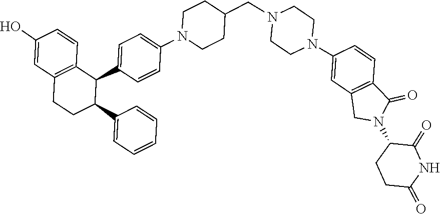 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

is 200 mg.

In embodiments, the daily dose of Compound A:

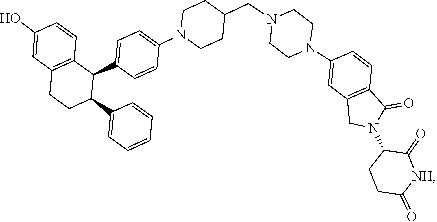 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

is 100 mg.

In embodiments, Compound A:

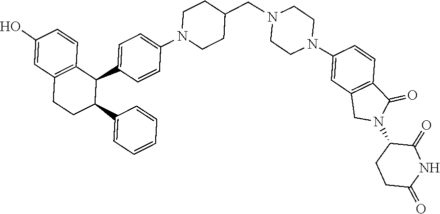 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

is administered as a free base.

In embodiments, the cancer is breast cancer, lung cancer, colon cancer, brain cancer, head and neck cancer, prostate cancer, stomach cancer, pancreatic cancer, ovarian cancer, melanoma, endocrine cancer, uterine cancer, testicular cancer, or bladder cancer.

In embodiments, the cancer is breast cancer, lung cancer, prostate cancer, pancreatic cancer, or ovarian cancer.

In embodiments, the cancer is breast cancer, lung cancer, or prostate cancer.

In embodiments, the cancer is breast cancer.

In embodiments, the breast cancer is metastatic or locally advanced.

In embodiments, the breast cancer is estrogen receptor positive (ER+) breast cancer.

In embodiments, the estrogen receptor positive (ER+) breast cancer is human epidermal growth factor receptor 2 negative (HER2−).

In embodiments, the subject is human.

Also disclosed herein is Compound A:

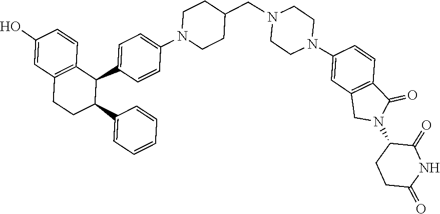 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

or a pharmaceutically acceptable salt thereof, for use according to any one of foregoing embodiments.

Also disclosed herein are uses of Compound A:

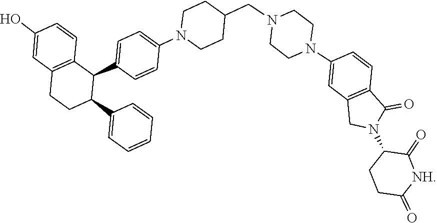 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

or a pharmaceutically acceptable salt thereof, in the manufacture of a medicament according to any one of foregoing embodiments.

Each of the embodiments described herein may be combined with any other embodiment(s) described herein not inconsistent with the embodiment(s) with which it is combined.

### Administration and Dosing

The terms “treat” and “treating” a cancer or a cancer-associated disease, as used herein, mean to administer a monotherapy according to the present disclosure to a subject, participant or patient having a cancer, or diagnosed with a cancer, to achieve at least one positive therapeutic effect, such as, for example, reduced number of cancer cells, reduced tumor size, reduced rate of cancer cell infiltration into peripheral organs, or reduced rate of tumor metastasis or tumor growth, reversing, alleviating, inhibiting the progress of, or preventing the disorder or condition to which such term applies, or one or more symptoms of such disorder or condition. The terms “treatment” and “therapy,” as used herein, unless otherwise indicated, refer to the act of treating as “treating” is defined immediately above. For the purposes of this disclosure, beneficial or desired clinical results include, but are not limited to, one or more of the following: reducing the proliferation of (or destroying) neoplastic or cancerous cell; inhibiting metastasis or neoplastic cells; shrinking or decreasing the size of tumor; remission of the cancer; decreasing symptoms resulting from the cancer; increasing the quality of life of those suffering from the cancer; decreasing the dose of other medications required to treat the cancer; delaying the progression the cancer; curing the cancer; overcoming one or more resistance mechanisms of the cancer; and/or prolonging survival of patients the cancer. Positive therapeutic effects in cancer can be measured in a number of ways (see, for example, W. A. Weber, J. Nucl. Med. (2009) 50:1S-10S).

“Fed condition” or “fed state” as used to describe a subject herein, means that the subject has eaten less than 4 hours before a time point of interest, such as the time of administering Compound A. In embodiments, a subject in the fed state has eaten within any of 4, 3, 2, 1, or 0.5 hours prior to administration of Compound A.

An “amount” for use and for treating a subject refers to an amount that provides, in single or multiple doses, alone, a detectable response of any duration of time (transient, medium or long term), a desired outcome in or an objective or subjective benefit to a subject of any measurable or detectable degree or for any duration of time (e.g., for hours, days, months, years, in remission or cured). Such amounts typically are effective to ameliorate a disease, or one, multiple or all adverse effects/symptoms, consequences, or complications of the disease, to a measurable extent, although reducing or inhibiting a progression or worsening of the disease, or providing stability (i.e., not worsening) state of the disease, is considered a satisfactory outcome. The term “therapeutically effective amount” also means an amount of an agent, alone, effective for producing a desired therapeutic effect upon administration to a subject, for example, to stem the growth, or result in the shrinkage, of a cancerous tumor. In reference to the treatment of cancer, a therapeutically effective amount refers to that amount that has the effect of (1) reducing the size of the tumor, (2) inhibiting (that is, slowing to some extent, preferably stopping) tumor metastasis emergence, (3) inhibiting to some extent (that is, slowing to some extent, preferably stopping) tumor growth or tumor invasiveness, and/or (4) relieving to some extent (or, preferably, eliminating) one or more signs or symptoms associated with the cancer. Therapeutic or pharmacological effectiveness of the doses and administration regimens also may be characterized as the ability to induce, enhance, maintain, or prolong disease control and/or overall survival in patients with these specific tumors, which may be measured as prolongation of the time before disease progression.

As used herein, “ameliorate” refers to any reduction in the extent, severity, frequency, and/or likelihood of a symptom or clinical sign characteristic of a particular disease. “Symptom” refers to any subjective evidence of disease or of a subject's condition.

Embodiments of the present invention provide a dose, dosage, and dosing regimen comprising administering to a subject an amount, or an effective amount of Compound A, or a pharmaceutically acceptable salt thereof. The amount, or the therapeutically effective amount, can be a daily dose of about 200 mg of the free base of Compound A, or an equivalent amount of a pharmaceutically acceptable salt thereof. In embodiments, a daily dose is 200 mg of the free base of Compound A, or an equivalent amount of a pharmaceutically acceptable salt thereof.

Embodiments of the present invention provide a dose, dosage, and dosing regimen comprising administering to a subject an amount, or an effective amount of Compound A, or a pharmaceutically acceptable salt thereof. The amount, or the therapeutically effective amount, can be a daily dose of about 100 mg of the free base of Compound A, or an equivalent amount of a pharmaceutically acceptable salt thereof. In embodiments, a daily dose is 100 mg of the free base of Compound A, or an equivalent amount of a pharmaceutically acceptable salt thereof.

In embodiments, the daily dose of Compound A, or a pharmaceutically acceptable salt thereof, is administered once per day (QD).

Compound A, or a pharmaceutically acceptable salt thereof, may be administered orally. Oral administration may involve swallowing, so that Compound A enters the gastrointestinal tract, or buccal or sublingual administration may be employed by which Compound A enters the bloodstream directly from the mouth.

In embodiments, the daily dose of Compound A, or a pharmaceutically acceptable salt thereof, is administered orally.

Compound A, or a pharmaceutically acceptable salt thereof, may be present in a pharmaceutical composition, which includes a pharmaceutically acceptable excipient. A “pharmaceutically acceptable excipient” refers to a component that may be included in the compositions described herein, is physiologically suitable for pharmaceutical use, and causes no significant adverse effects nor therapeutic effects to a subject. The term “excipient” is used herein to describe any ingredient other than Compound A, or a pharmaceutically acceptable salt thereof. The choice of excipient will to a large extent depend on factors such as the mode of administration, the effect of the excipient on solubility and stability, and the nature of the dosage form.

Compound A of the methods and uses of the present invention may be formulated prior to administration. The formulation preferably will be adapted to the particular mode of administration. Compound A, or a pharmaceutically acceptable salt thereof, may be formulated with pharmaceutically acceptable excipients as known in the art and administered in a wide variety of dosage forms as known in the art. Dosage unit forms or pharmaceutical compositions suitable for oral administration include, but are not limited to tablets, capsules, such as gelatin capsules, pills, powders, granules, aqueous, and nonaqueous oral solutions and suspensions, packaged in containers adapted for subdivision into individual doses.

Repetition of the administration or dosing regimens may be conducted as necessary to achieve the desired reduction or diminution of cancer cells. A “continuous dosing schedule,” as used herein, is an administration or dosing regimen without dose interruptions, e.g., without days off treatment. Repetition of 28-day treatment cycles without dose interruptions between the treatment cycles is an example of a continuous dosing schedule.

In embodiments, Compound A, or a pharmaceutically acceptable salt thereof, is administered once daily to comprise a complete cycle of 28-days. Repetition of 28-day treatment cycles is continued during treatment in accordance with the methods and uses of the present disclosure.

### Methods of Treatment

In embodiments, provided herein are methods for treating cancer in a subject, comprising administering to the subject an effective amount of Compound A, or a pharmaceutically acceptable salt thereof, as described herein.

The term “locally advanced,” as used herein, as it relates to cancer, may or may not be treated with curative intent. For example, locally advanced breast cancer (LABC) is defined by the U.S. National Comprehensive Cancer Network as a subset of breast cancer characterized by the most advanced breast tumors in the absence of distant metastasis, wherein the tumors are more than 5 cm in size with regional lymphadenopathy; tumors of any size with direct extension to the chest wall or skin, or both (including ulcer or satellite nodules), regardless of regional lymphadenopathy; presence of regional lymphadenopathy (clinically fixed or matted axillary lymph nodes, or any of infraclavicular, supraclavicular, or internal mammary lymphadenopathy) regardless of tumor stage. (Garg et al. Curr Oncol. 2015 October; 22(5): e409-e410; National Comprehensive Cancer Network NCCN Clinical Practice Guidelines in Oncology: Breast Cancer. Fort Washington, PA: NCCN; 2015. Ver. 2.2015.)

The term “metastatic” as used herein, as it relates to cancer, cannot be treated with curative intent.

For example, metastatic breast cancer refers to breast cancer that has spread beyond the breast and nearby lymph nodes to other parts of the body, e.g., bones, liver, lungs, brain. (www.cancer.org/cancer/breast-cancer.) Those skilled in the art will be able to recognize and diagnose locally advanced and metastatic cancer in a patient or subject.

For convenience, certain well-known abbreviations, may be used herein, including: castration resistant prostate cancer (CRPC), estrogen receptor positive (ER+), human epidermal growth factor receptor 2 negative (HER2−), hormone receptor (HR), human epidermal growth factor receptor 2 positive (HER2+), non-small cell lung cancer (NSCLC), and progesterone receptor (PR).

In embodiments, the cancer is selected from lung cancer, mesothelioma, bone cancer, pancreatic cancer, skin cancer, cancer of the head or neck, cutaneous or intraocular melanoma, uterine cancer, ovarian cancer, rectal cancer, cancer of the anal region, stomach cancer, hepatic carcinoma, colon cancer, breast cancer, uterine cancer, carcinoma of the fallopian tubes, carcinoma of the endometrium, carcinoma of the cervix, carcinoma of the vagina, carcinoma of the vulva, Hodgkin's disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, hematology malignancy, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, glioblastoma, brain stem glioma, pituitary adenoma, head and neck cancer, and combinations of two or more of the foregoing cancers.

Also disclosed herein are methods of treating cancer in a subject. In embodiments, the methods comprise treating cancer in a subject comprising administering to the subject an amount of Compound A, or a pharmaceutically acceptable salt thereof, described herein that are effective in treating the cancer.

In embodiments, the cancer is breast cancer, lung cancer, colon cancer, brain cancer, head and neck cancer, prostate cancer, stomach cancer, pancreatic cancer, ovarian cancer, melanoma, endocrine cancer, uterine cancer, testicular cancer, or bladder cancer.

In embodiments, the cancer is breast cancer, lung cancer, prostate cancer, pancreatic cancer, or ovarian cancer.

In embodiments, the cancer is breast cancer, lung cancer, or prostate cancer.

In embodiments, the cancer is breast cancer.

In embodiments, the breast cancer is metastatic breast cancer.

In embodiments, the breast cancer is locally advanced breast cancer.

In embodiments, the breast cancer is HR+ breast cancer.

In embodiments, the HR+ breast cancer is PR+ and/or ER+ breast cancer.

In some embodiments, the breast cancer is PR+ breast cancer.

In embodiments, the breast cancer is ER+ breast cancer.

In embodiments, the breast cancer is ER+ HER2− breast cancer.

In embodiments, the breast cancer is ER+ HER2+ breast cancer.

In embodiments, the breast cancer is locally advanced or metastatic ER+ breast cancer.

In embodiments, the breast cancer is locally advanced or metastatic ER+ HER2− breast cancer.

In embodiments, the breast cancer is locally advanced or metastatic ER+ HER2+ breast cancer.

In embodiments, the breast cancer is metastatic, ER+, HER2− breast cancer.

In embodiments, the breast cancer is metastatic, ER+, HER2− breast cancer that is also locally advanced.

In embodiments, the lung cancer is non-small cell lung cancer.

In embodiments, the lung cancer is locally advanced or metastatic non-small cell lung cancer.

In embodiments, the prostate cancer is CRPC.

In embodiments, the prostate cancer is locally advanced or metastatic CRPC.

Also disclosed herein are methods of treating solid tumors in a subject. In embodiments, disclosed herein are methods of treating solid tumors in a subject comprising administering to the subject an amount of Compound A, or a pharmaceutically acceptable salt thereof, effective in treating the solid tumor.

In embodiments, the solid tumor is breast cancer, lung cancer, colon cancer, brain cancer, head and neck cancer, prostate cancer, stomach cancer, pancreatic cancer, ovarian cancer, melanoma, endocrine cancer, uterine cancer, testicular cancer, or bladder cancer.

In embodiments, the solid tumor is breast cancer, lung cancer, prostate cancer, pancreatic cancer, or ovarian cancer.

In embodiments, the solid tumor is breast cancer, lung cancer, or prostate cancer.

In embodiments, the solid tumor is breast cancer. For example, in certain embodiments the breast cancer is HR+ breast cancer. In other embodiments, the HR+ breast cancer is PR+ and/or ER+ breast cancer ER+ breast cancer.

In embodiments, the solid tumor is breast cancer. For example, in certain embodiments, the breast cancer is ER+ HER2− breast cancer.

In embodiments, the solid tumor is breast cancer. For example, in certain embodiments, the breast cancer is ER+ HER2+ breast cancer.

In embodiments, the solid tumor is breast cancer. For example, in certain embodiments, the breast cancer is locally advanced or metastatic ER+ HER2− breast cancer.

In embodiments, the solid tumor is breast cancer. For example, in certain embodiments, the breast cancer is locally advanced or metastatic ER+ HER2+ breast cancer.

In embodiments, the solid tumor is lung cancer. For example, in certain embodiments, the lung cancer is non-small cell lung cancer.

In embodiments, the solid tumor is lung cancer. For example, in certain embodiments, the lung cancer is locally advanced or metastatic non-small cell lung cancer.

In embodiments, the solid tumor is prostate cancer. For example, in certain embodiments, the prostate cancer is CRPC.

In embodiments, the solid tumor is prostate cancer. For example, in certain embodiments, the prostate cancer is locally advanced or metastatic castration resistant prostate cancer.

Also disclosed herein are methods of treating hematologic tumors in a subject. In certain embodiments, the method comprises treating hematologic tumors in a subject comprising administering to the subject an amount of Compound A, or a pharmaceutically acceptable salt thereof, that is effective in treating the hematologic tumor.

In embodiments, the hematologic tumor is leukemia, lymphoma, or multiple myeloma.

In embodiments, the hematologic tumor is leukemia or lymphoma.

Also disclosed herein are methods of treating cancer in a subject with locally advanced or metastatic ER+ HER2− breast cancer, CRPC, or NSCLC whose disease progressed on or is intolerant to standard therapy.

Also disclosed herein are methods of treating cancer in a subject with locally advanced or metastatic ER+ HER2− breast cancer, CRPC, or NSCLC whose disease progressed on or is intolerant to standard therapy.

Also disclosed herein are methods of treating cancer in a subject with locally advanced or metastatic 2L+ER+ HER2 breast cancer who have received prior hormonal/endocrine therapy and chemotherapy in the locally advanced/metastatic setting. In embodiments, the method comprises administering Compound A, or a pharmaceutically acceptable salt thereof, as a single agent.

Also disclosed herein are methods of treating cancer in a subject with locally advanced or metastatic 2L+ER+HER2 breast cancer who have received prior treatment with a CDK4/6 inhibitor. In embodiments, the method comprises administering Compound A, or a pharmaceutically acceptable salt thereof, as a single agent.

### EXAMPLES

In order that this invention may be better understood, the following examples are set forth. These examples are for purposes of illustration only and are not to be construed as limiting the scope of the invention in any manner.

Abbreviations used herein include:
AUC₀₋₈=area under the plasma concentration-time curve from zero to 8 hours;
AUC₀₋₁₂=area under the plasma concentration-time curve from 0 to 12 hours;
AUCₜₐᵤ=area under the plasma concentration-time curve over the dosing interval, where tau is 24 hours for once daily dosing and 12 hours for twice daily dosing;
BID=twice daily;
BMI=body mass index;
CI=confidence interval;
CR=complete response
CBR=clinical benefit rate;
CL/F=oral clearance
Cₘₐₓ=maximum plasma concentration;
Tₘₐₓ=time to maximum concentration;
DDI=drug-drug interaction;
ER=estrogen receptor;
GCV=geometric coefficient of variation;
HER2=human epidermal growth factor receptor 2;
FIH=first-in-human;
IR=immediate-release;
mBC=metastatic breast cancer;
MTD=maximum tolerated dose;
ORR=overall response rate;
PPI=proton pump inhibitor;
PR=partial response
rBA=relative bioavailability;
RP2D=recommended Phase 2 dose;
R_{AC}=drug accumulation ratio
QD=once daily;
SA=single agent;
SD=stable disease;
SAR=serious adverse reaction;
SUSAR=suspected unexpected serious adverse reaction;
AE=adverse event;
SAE=serious adverse event;
TEAE=treatment-emergent adverse event;
TRAE=treatment-related adverse event-; and
V_{SS}=volume of distribution at steady state.

### Compound a Clinical Trials (“the FIH Study” and “the Clin Pharm Study”) The First-In-Human (“FIH”) Study

Compound A is being investigated in an ongoing Phase 1/2, open label, dose escalation and cohort expansion study to evaluate the safety, tolerability, pharmacokinetics and anti-tumor activity of Compound A alone in patients with estrogen receptor positive/human epidermal growth factor receptor 2 negative (ER+/HER2−) locally advanced or metastatic breast cancer, who have received prior hormonal therapy and chemotherapy in the locally advanced/metastatic setting. An overview of the FIH Study (Parts A and B) is provided in Table 1 below.

The FIH Study (Parts A and B) will assess the safety and tolerability, and determine the MTD and/or RP2D, of Compound A, either as a single agent, in patients with locally advanced or mBC. The study will also assess clinical activity of Compound A at the RP2Ds for monotherapy. Additional evaluation will include single- and multiple-dose pharmacokinetics and biochemical activity.

Study Design: In the FIH Study: Part A is a Phase I monotherapy dose escalation, and Part B is a Phase 2 monotherapy expansion at 2 RP2D doses (200 mg QD, 500 mg QD).

### Method of Administration

Compound A was orally administered as monotherapy (Part A and B), in 28-day cycles, in doses shown in Table 1. Compound A was supplied as 10 mg, 50 mg, and 100 mg strength immediate release tablets. Tableting excipients were inert, compendial components commonly employed in oral formulations, including lactose monohydrate and sodium stearyl fumarate.

TABLE 1
Overview of the FIH Study of Parts A and Parts B

| | | | Compound A Treatment |
|---|---|---|---|
| Study Title | Study Design | Study Population | Groups Evaluated |
| A Phase 1/2, open | FIH, | Patients with | Part A - Phase 1 Dose |
| label, dose escalation, | open-label, | histologically or | Escalation: |
| and cohort expansion | sequential | cytologically confirmed | 30 mg QD |
| clinical trial to evaluate | group, dose | ER+/HER2− metastatic, | 60 mg QD |
| the safety, tolerability, | escalation 3 + 3 | recurrent, or locally | 120 mg QD |
| and pharmacokinetics | and cohort | advanced unresectable | 180 mg QD^{a} |
| of Cpd A alone and in | expansion | breast cancer for which | 360 mg QD^{b} |
| combination with | consisting of 3 | standard curative | 500 mg QD (or 250 mg BID) |
| palbociclib | parts: | therapy is no longer | 700 mg QD (or 400 mg |
| (IBRANCE ®) in | Part A - Phase | effective or does not | AM/300 mg PM) |
| patients with estrogen | 1 dose | exist | Part B - Phase 2 Expansion as |
| receptor positive/human | escalation | Age: ≥18 years | SA |
| epidermal growth factor | Part B - Phase | | 200 mg QD |
| receptor 2 negative | 2 cohort | | 500 mg QD |
| (ER+/HER2−) locally | expansion: | | |
| advanced or metastatic | RP2D | | |
| breast cancer, who have | | | |
| received prior hormonal | | | |
| therapy and | | | |
| chemotherapy in the | | | |
| locally | | | |
| advanced/metastatic | | | |
| setting | | | |

^{a}Patients initially enrolled into this cohort received 180 mg QD, but the dose was rounded up to 200 mg QD upon availability of the 100 mg tablets.
^{b}Patients initially enrolled into this cohort received 360 mg QD, but the dose was rounded up to 400 mg QD upon availability of the 100 mg tablets

### The Clinical Pharmacology Study

Compound A is being investigated in an ongoing Phase 1 clinical pharmacology study to evaluate the effect of food or a PPI (esomeprazole) on the single-dose PK and safety of Compound A in healthy postmenopausal female volunteers (“participants”). An overview of the Clinical Pharmacology Study is provided in Table 2 below.

Study Design: The Clinical Pharmacology Study consists of 3 independent cohorts: 1) an open-label, randomized, 2-period, crossover Fed/Fasted cohort to determine food effects; 2) an open-label, 2-period, fixed sequence PPI cohort to evaluate interactions with esomeprazole; and 3) an open-label, randomized, 2 period, crossover rBA cohort to evaluate 2 tablet formulations.

As of the data cut-off date of 16 Jun. 2022, 47 participants have been treated in the 3 cohorts of Clinical Pharmacology Study. (14 participants in Fed/Fasted, 17 participants in PPI, and 16 participants in rBA cohorts). Each participant received 2 doses of 200 mg Compound A, 1 in each of the 2 periods, and the Compound A treatments were separated by a washout period of at least 14 days.

A dose of 200 mg Compound A has been well-tolerated in the participants, and no TRAEs of Grade 2 or higher were reported.

TABLE 2
Overview of the Clinical Pharmacology Study

| | | Study | |
|---|---|---|---|
| Study Title | Study Design | Population | Compound A Treatment Groups Evaluated |
| A Phase 1, | Open-label study | Healthy | Each dose cohort receives 200 mg Cpd A |
| multi-part, | with 3 independent | post- | following a 10-hour overnight fast. |
| open-label study | parts: | menopausal | Fed/Fasted Cohort: Cpd A dosing on Day 1 |
| to evaluate the | Fasted/Fed: | women with | and Day 15: |
| effect of food or | 2-period, crossover | BMI 18.5 to | Fasted Period: fasting continues for at least |
| a proton-pump | Fed/Fasted cohort | 35.0 kg/m² | 4 hours after dosing |
| inhibitor | to determine food | and body | Fed Period: Cpd A administered 30 min |
| (esomeprazole) | effects | weight 50 to | after starting to eat a high-fat meal, then |
| and to evaluate | PPI: | 120 kg, | fasting for at least 4 hours after Cpd A |
| the relative | 2-period, fixed | inclusive. | administration |
| bioavailability of | sequence PPI | Age: 18 to | PPI Cohort: |
| different tablet | cohort to evaluate | 70 years | Single-dose period: Cpd A administered in a |
| formulations on | interactions with | | fed state^{c} on Day 1 and Day 19 |
| the single-dose | esomeprazole | | Sequential-dose period: esomeprazole |
| pharmacokinetics | rBA: | | delayed-release capsule (40 mg QD) at least |
| and safety of Cpd | randomized, 2- | | 1 hour prior to food for 5 days from Days |
| A in healthy | period, crossover | | 15-19. On Day 19 at least 90 min after |
| post-menopausal | rBA cohort to | | esomeprazole, Cpd A is administered in a |
| female volunteers | evaluate 2 tablet | | fed state^{d} |
| | formulations | | rBA Cohort: |
| | | | 200 mg Cpd A administered as either 20% |
| | | | (reference) or 42% (test) drug load on Day 1 |
| | | | and Day 15 in a fed state^{c} |

^{c}Study drug administered 30 minutes after starting to eat a moderate-fat meal, then fasting for at least 4 hours after Compound A administration.

### Example 1: Pharmacokinetics (PK) in Patients with Breast Cancer (the FIH Study)

Preliminary PK data from Part A monotherapy dose escalation of the FIH Study for Compound A were available from dose levels ranging from 30 to 700 mg administered either as QD or BID. Preliminary results indicated dose-dependent increases in Cₘₐₓ and AUCₜₐᵤ for Compound A, Compound B, and sum of Compound A+Compound B up to 500 mg total daily dose administered either as 250 mg BID or 500 mg QD on both Cycle 1 Day 1 and Day 15 (Tables 3-5). The median Tₘₐₓ ranged from 4 to 7 hours across the dose levels. The mean effective T1/2 at steady state ranged from 23 to 33 hours.

Geometric mean Compound A AUCₜₐᵤ was 7324 ng·h/mL on Day 15 at 60 mg QD (N=3). Following 200 mg QD dosing (N=8), a geometric mean accumulation ratio based on AUCₜₐᵤ of 1.7 was observed between Day 1 and Day 15. The ratio of Compound B/Compound A, based on AUCₜₐᵤ, on Cycle 1 Day 15 is 32%.

TABLE 3
Preliminary Compound A Pharmacokinetic Data of the FIH Study after 1st dose
(Day 1 of Cycle 1) and at steady state (Day 15 of Cycle 1) (Data Cut-off Date of
6 Jun. 2022)

| | Compound A | | | | | | | | | | |
|---|---|---|---|---|---|---|---|---|---|---|---|
| | Geometric Mean (GCV %) | | | | | | | | | | |
| | Day 1 | | | | Day 15 | | | | | | |
| | (Single Dose) | | | | (Multiple Dose) | | | | | | |
| | | AUCₜₐᵤ | | | | AUCₜₐᵤ | | | | | |
| | | (ng · | Cₘₐₓ | Tₘₐₓ | | (ng · | Cₘₐₓ | Tₘₐₓ | R_{ac} | t_{1/2,eff} | CL/F |
| Dose | N | h/mL) | (ng/mL) | (h) | N | h/mL) | (ng/mL) | (h) | (AUCₜₐᵤ) | (h) | (L/h) |
| 30 mg QD | 3 | 1713 | 109 | 4.0 | 3 | 4051 | 219 | 4.1 | 2.6 | 31 | 7.4 |
| | | (7%) | (6%) | (4.0- | | (26%) | (27%) | (3.9- | (1.9- | (8.2) | (26) |
| | | | | 5.9) | | | | 6.0) | 2.8) | | |
| 60 mg QD | 3 | 2964 | 194 | 6.0 | 3 | 7324 | 405 | 4.2 | 2.6 | 33 | 8.2 |
| | | (33%) | (33%) | (4.0- | | (15%) | (8%) | (4.0- | (2- | (7.6) | (15) |
| | | | | 6.1) | | | | 6.0) | 2.9) | | |
| 100 mg QD | 8 | 4146 | 221 | 5.8 | 7 | 8898 | 522 | 6.0 | 2.1 | 29 | 11.2 |
| | | (57%)^{c} | (87%) | (3.8- | | (39) | (46%) | (3.8- | (2.0- | (7.1) ^{e1} | (39) |
| | | | | 6.0) | | | | 8.0) | 3.1)^{e1} | | |
| 120 mg QD | 7 | 6326 | 404 | 5.9 | 7 | 13714 | 799 | 5.9 | 2.2 | 27 | 8.8 |
| | | (21%) | (21%) | (4.0- | | (13%) | (6%) | (3.9- | (1.8- | (7.5) | (13) |
| | | | | 7.8) | | | | 7.8) | 3.1) | | |
| 180 mg QD | 7 | 9389 | 555 | 6.0 | 6 | 19225 | 1062 | 5.0 | 2.3 | 26 | 9.4 |
| | | (16%) | (15%) | (2.0- | | (32%)^{e1} | (27%) | (2.1- | (1.5- | (8.3) | (32) |
| | | | | 7.8) | | | | 6.0) | 2.5) | | |
| 200 mg QD | 8 | 9114 | 534 | 5.8 | 8 | 15459 | 866 | 5.0 | 1.9 | 23 | 12.9 |
| | | (48%) | (52%) | (4.0- | | (34%) | (40%) | (3.8- | (0.9- | (12)^{c} | (34) |
| | | | | 7.8) | | | | 7.9) | 2.8) | | |
| 360 mg QD | 15 | 12172 | 748 | 5.8 | 15 | 25931 | 1502 | 5.8 | 2.2 | 28 | 13.9 |
| | | (32%) | (34%) | (4.0- | | (27%) | (27%) | (3.9- | (1.5- | (11) | (27) |
| | | | | 7.8) | | | | 8.1) | 3.5) | | |
| 500 mg QD | 12 | 16765 | 1046 | 4.9 | 11 | 34585 | 1883 | 5.9 | 2.2 | 28 | 14.5 |
| | | (30%) ^{d} | (31%) | (1.0- | | (35%) | (31%) | (3.8- | (1.1- | (15) ^{f} | (36) |
| | | | | 8.0) | | | | 7.8) | 3.8) ^{f} | | |
| 700 mg QD | 1 | 19639 ^{e} | 1450 ^{e} | 5.8 ^{g} | 1 | 26572 ^{e} | 1530 ^{e} | 6.0 g | 1, 4 ^{g} | 12.4 ^{g} | 26.3 ^{g} |
| 250 mg BID | 10 | 2924 | 663 | 6.9 | 8 | 20559 | 2058 | 5.1 | — | — | 12.2 |
| (500 mg total | | (32%)ª | (18%) | (2.0- | | (34%)^{b} | (34%) | (1.9- | | | (34) |
| daily dose) | | | | 7.9) | | | | 7.9) | | | |
| 400 mg | 3 | 3798 | 716 | 4.1 | 2 | 35360, | 3360, | 4.1, | — | — | 11.3, |
| AM/300 mg | | (102%)^{a} | (131%) | (3.9- | | 14815^{b,e1} | 1510^{e1} | 6.0 ^{g} | | | 27 ^{g} |
| PM (700 | | | | 5.8) | | | | | | | |
| mg total | | | | | | | | | | | |
| daily dose) | | | | | | | | | | | |

^{a}AUC₀₋₈
^{b}AUC₀₋₁₂
^{c}N = 7
^{d} N = 11
^{e1}N = 6
^{e} Expressed as individual values when N < 3.
^{f} N = 10
^{g} Expressed as individual values when N < 3.

TABLE 4
Preliminary Compound B Pharmacokinetic Data of the FIH Study after 1st dose
(Day 1 of Cycle 1) and at steady state (Day 15 of Cycle 1) (Data Cut-off Date of
6 Jun. 2022)

| | Compound B | | | | | | | | | | | |
|---|---|---|---|---|---|---|---|---|---|---|---|---|
| | Geometric Mean (GCV %) | | | | | | | | | | | |
| | Day 1 | | | | | Day 15 | | | | | | |
| | (Single Dose) | | | | | (Multiple Dose) | | | | | | |
| | | AUCtau | Cₘₐₓ | | | | AUCₜₐᵤ | Cₘₐₓ | | | | |
| | | (ng · h/ | (ng/ | Tₘₐₓ | MR | | (ng · h/ | (ng/ | Tₘₐₓ | R_{ac} | MR | t_{1/2,eff} |
| Dose | N | mL) | mL) | (h) | AUCₜₐᵤ | N | mL) | mL) | (h) | (AUCₜₐᵤ) | AUCₜₐᵤ | (h) |
| 30 mg QD | 3 | 194 | 8 | 8 | 0.11 | 3 | 1279 | 58 | 6.0 | 7.1 | 0.32 | 104 |
| | | (4%) | (4%) | (8.0- | (3) | | (33%) | (32%) | (3.9 - | (4.8 - | (10) | (30) |
| | | | | 8.1) | | | | | 7.8) | 8.4) | | |
| 60 mg QD | 3 | 323 | 13 | 7.9 | 0.11 | 3 | 2243 | 108 | 2.1 | 7.3 | 0.31 | 110 |
| | | (36%) | (38%) | (7.9- | (3) | | (8%) | (6%) | (1.0 - | (5.2 - | (8) | (31) |
| | | | | 8.0) | | | | | 5.9) | 8.9) | | |
| 100 mg QD | 8 | 443 | 17 | 7.8 | 0.11 | 7 | 2515 | 125 | 6.0 | 6.1 ^{f} | 0.28 | 99 ^{f} |
| | | (61%)^{c} | (111%) | (5.8 | (11) c | | (40%) | (46%) | (3.8 - | (4.9 - | (14) | (27) |
| | | | | 8.0) | | | | | 8.0) | 9.4) | | |
| 120 mg QD | 7 | 674 | 29 | 7.8 | 0.11 | 7 | 3974 | 191 | 6.0 | 5.7 | 0.29 | 93 |
| | | (20%) | (25%) | (5.9- | (14) | | (24%) | (19%) | (3.9 - | (4.4 - | (17) | (32) |
| | | | | 7.9) | | | | | 7.8) | 9.7) | | |
| 180 mg QD | 7 | 1014 | 40 | 7.8 | 0.11 | 6 | 5799 | 273 | 5.9 | 6.8 | 0.30 | 92 |
| | | (11%) | (24%) | (2.0- | (12) | | (34%) | (32%) | (3.9 - | (3.2 - | (14) | (32) |
| | | | | 7.8) | | | | | 7.8) | 8.0) | | |
| 200 mg QD | 8 | 959 | 40 | 7.8 | 0.11 | 8 | 4990 | 234 | 5.9 | 5.1 | 0.32 | 85 |
| | | (43%) | (41%) | (6.0- | (17) | | (35%) | (34%) | (1.0- | (3.1 - | (14) | (40) |
| | | | | 8.0) | | | | | 7.9) | 8.9) | | |
| 360 mg QD | 15 | 1352 | 60 | 7.8 | 0.11 | 15 | 8077 | 382 | 6.0 | 5.9 | 0.31 | 96 |
| | | (37%) | (34%) | (5.8- | (16) | | (34%) | (31%) | (2.0 - | (3.1 - | (15) | (33) |
| | | | | 8.0) | | | | | 8.8) | 9.8) | | |
| 500 mg QD | 12 | 1797 | 80 | 7.8 | 0.11 | 11 | 10198 | 480 | 6.0 | 5.9 | 0.30 | 96 |
| | | (28%)^{d} | (26%) | (4.0- | (8) d | | (41%) | (42%) | (3.8 - | (2.8- | (19) | (50) ^{e} |
| | | | | 8.0) | | | | | 7.8) | 12)^{e} | | |
| 700 mg QD | 1 | 2009^{g} | 92 ^{g} | 7.8 ^{g} | 0.10 ^{g} | 1 | 7387 ^{g} | 325 ^{g} | 7.8 ^{g} | 3.7 ^{g} | 0.28 ^{g} | 52 ^{g} |
| 250 mg BID | 10 | 179 | 52 | 7.8 | — | 8 | 7393 | 695 | 4.1 | — | 0.36 | — |
| (500 mg total | | (48%)^{a} | (26%) | (4.0- | | | (52%) ^{b} | (55%) | (1.0 - | | (26) | |
| daily dose) | | | | 8.1) | | | | | 7.9) | | | |
| 400 mg | 3 | 220 | 47 | 7.8 | — | 2 | 12334, | 1060, | 5.9, | — | 0.26, | — |
| AM/300 mg | | (96%)^{a} | (119%) | (5.8- | | | 3791 ^{b,g} | 378 ^{g} | 6.0 ^{g} | | 0.35 ^{g} | |
| PM (700 | | | | 7.9) | | | | | | | | |
| mg total | | | | | | | | | | | | |
| daily dose) | | | | | | | | | | | | |

^{a}AUC₀₋₈
^{b} AUC₀₋₁₂
^{c} N = 7
^{d} N = 11
^{e} N = 10
^{f} N = 6
^{g} Expressed as individual values when N < 3.

TABLE 5
Preliminary Compound A + Compound B Pharmacokinetic Data
of the FIH Study after 1st dose (Day 1 of Cycle 1) and at steady
state (Day 15 of Cycle 1) (Data Cut-off Date of 6 Jun. 2022)

| | Compound A + Compound B | | | | | |
|---|---|---|---|---|---|---|
| | Geometric Mean (GCV %) | | | | | |
| | Day 1 | | | Day 15 | | |
| | (Single Dose) | | | (Multiple Dose) | | |
| | | AUCₜₐᵤ | Cₘₐₓ | | AUCₜₐᵤ | Cₘₐₓ |
| | | (ng · h/ | (ng/ | | (ng · h/ | (ng/ |
| Dose | N | mL) | mL) | N | mL) | mL) |
| 30 mg QD | 3 | 1907 | 116 | 3 | 5334 | 276 |
| | | (6%) | (5%) | | (27%) | (28%) |
| 60 mg QD | 3 | 3287 | 206 | 3 | 9570 | 500 |
| | | (34%) | (33%) | | (14%) | (6%) |
| 100 mg QD | 8 | 4590 | 236 | 7 | 11431 | 648 |
| | | (57%)^{c} | (88%) | | (39%) | (45%) |
| 120 mg QD | 7 | 7005 | 430 | 7 | 17727 | 991 |
| | | (21%) | (21%) | | (15%) | (6%) |
| 180 mg QD | 7 | 10408 | 593 | 6 | 25059 | 1334 |
| | | (15%) | (14%) | | (32%) | (27%) |
| 200 mg QD | 8 | 10084 | 569 | 8 | 20480 | 1091 |
| | | (47%) | (52%) | | (34%) | (38%) |
| 360 mg QD | 15 | 13540 | 799 | 15 | 34074 | 1875 |
| | | (32%) | (34%) | | (28%) | (26%) |
| 500 mg QD | 12 | 18567 | 1116 | 11 | 44908 | 2360 |
| | | (30%)^{d} | (31%) | | (36%) | (32%) |
| 700 mg QD | 1 | 21649 ^{e} | 1532 ^{e} | 1 | 33960 ^{e} | 1845 ^{e} |
| 250 mg BID | 10 | 3106 | 709 | 8 | 28120 | 2733 |
| (500 mg total | | (33%)^{a} | (18%) | | (38%)^{b} | (39%) |
| daily dose) | | | | | | |
| 400 mg | 3 | 4018 | 757 | 2 | 47694, | 4360, |
| AM/300 mg | | (101%)^{a} | (130%) | | 18605^{b,e} | 1888 ^{e} |
| PM (700 | | | | | | |
| mg total | | | | | | |
| daily dose) | | | | | | |

^{a}AUC₀₋₈
^{b}AUC₀₋₁₂
^{c}N = 7
^{d}N = 11
^{e} Expressed as individual values when N < 3.

### Pharmacokinetics in Healthy Adult Participants

### Pilot Food Effect Study

A Fed/Fasted Cohort was conducted to characterize the effect of food on the single dose PK of Compound A 200 mg in healthy participants. Fourteen participants were dosed with Compound A 200 mg in a fasted and fed state (high-fat meal of 800-1000 calories, consisting of at least 150 calories from protein, 250 calories from carbohydrates and 500-600 calories from fat). High-fat meal increased Compound A Cₘₐₓ 2.8-fold and AUC_{inf} 2.2-fold respectively. Compound A should be taken with food.

### Relative Bioavailability Study to Compare the Previous Clinical Formulation and The Phase 3 Formulation

To evaluate the relative bioavailability of Compound A phase 3 tablet formulation (100 mg strength) compared with the previous clinical formulation (100 mg strength) with a moderate-fat meal (approximately 700 calories with a fat content of approximately 35%) in 16 healthy participants. Compound A was dosed as 200 mg single dose (2×100 mg tablets). The geometric mean ratios for Cₘₐₓ and AUC_{inf} (90% CI) were 94.4% (83.4%, 106.9%) and 90.7% (84.8%, 97.2%), respectively. The 2 tablet formulations of Compound A demonstrated similar PK profiles under fed conditions with a moderate-fat meal and met the commonly accepted bioequivalence criteria (90% CI of 80% to 125%).

### Other Clinical Pharmacology Data Pharmacodynamic Effects in Humans

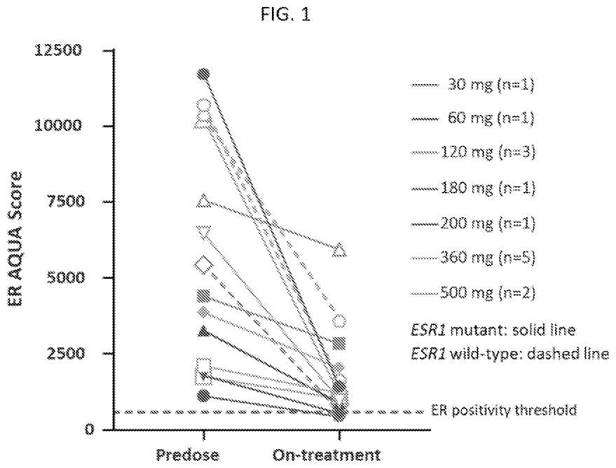

Exploratory analysis of ER expression in 14 paired biopsies from patients treated in various Part A dose escalation cohorts indicated ER degradation (median decrease=67% [range: 21% to 89%]) at all doses of Compound A tested (up to 500 mg QD) and in tumors expressing either WT or mutant ER proteins (FIG. 1). No relationship between ER degradation and dose or exposure has been observed to date.

### Example 2: Pharmacokinetics (PK) in Healthy Volunteers (the Clinical Pharmacology Study)

In the Clinical Pharmacology Study, preliminary PK parameters following a single 200 mg Compound A dose from all 3 cohorts were calculated. The median Tₘₐₓ ranged from 6.0 to 8.0 hours across the cohorts. The geometric mean T1/2 following a single 200 mg dose was approximately 40 hours under fed condition.

To characterize the effect of food on the single dose PK of Compound A (200 mg), fourteen healthy participants were administered with 200 mg Compound A in a fasted and fed state (high-fat meal of 800-1000 calories, consisting of at least 150 calories from protein, 250 calories from carbohydrates and 500-600 calories from fat). High-fat meal increased Compound A Cₘₐₓ and AUC_{inf} by 184% and 125%, respectively.

Statistical analysis showed that food intake significantly increased Compound A Cₘₐₓ and AUC_{inf} 3- to 2-fold, respectively, as compared with fasted conditions. Therefore, patients should be instructed to take Compound A with food.

Compound A AUC values were similar when administered with PPI or without PPI, although PPI decreased Compound A median Cₘₐₓ about 18% which is not considered clinical meaningful.

The effect of multiple doses of esomeprazole (40 mg) on the single dose PK of Compound A (200 mg) under fed conditions with a moderate-fat meal in 17 healthy participants was evaluated. Results showed that multiple doses of esomeprazole decreased Compound A exposure Cₘₐₓ by 17.1% and AUC_{inf} by 11%. These results indicated that under fed conditions with a moderate-fat meal (approximately 700 calories with a fat content of approximately 35%), multiple daily doses of a PPI (esomeprazole) had a minor effect on the extent of Compound A absorption. Without wishing to be bound by theory, this result suggests that there are no effects of PPI on exposure of Compound A when administered with a moderate-fat meal.

A relative bioavailability (rBA) cohort was conducted to evaluate the relative bioavailability of Compound A phase 3 tablet formulation (100 mg strength) compared with the previous clinical formulation (100 mg strength) with a moderate-fat meal (approximately 700 calories with a fat content of approximately 35%) in 16 healthy participants. Compound A was dosed as 200 mg single dose (2×100 mg tablets). The geometric mean ratios for Cₘₐₓ and AUC_{inf} (90% CI) were 94.4% (83.4%, 106.9%) and 90.7% (84.8%, 97.2%), respectively. The 2 tablet formulations of Compound A demonstrated similar PK profiles under fed conditions with a moderate-fat meal and met the commonly accepted bioequivalence criteria (90% CI of 80% to 125%).

### Example 3: Safety in Healthy Volunteers (the Clinical Pharmacology Study)

Adverse events (AEs) were coded to system organ class and preferred term according to the medical dictionary for regulatory activities (MedDRA), version 24.1. The severity of adverse events was graded according to the National Cancer Institute Common Terminology Criteria for Adverse Events (NCI CTCAE) version 5.0.

A treatment-emergent adverse event (TEAE) is an AE occurring after the first dose of Compound A and within 30 days of the last Compound A dose regardless of study drug attribution or grade.

A treatment-related adverse event (TRAE) is an AE assessed as “Possibly Related,” “Probably Related,” or “Related” to Compound A by the investigator.

### Treatment-Emergent Adverse Events (TEAEs)

Regardless of study drug attribution and grade, TEAEs were observed in 15 of the 47 participants treated with 200 mg Compound A in the ongoing Clinical Pharmacology Study (all cohorts).

Fast/Fed Cohort (n=14): The most common TEAE (regardless of attribution to study drug) observed in ≥10% of participants was ligament sprain (2 participants, 14.3%). All other TEAEs were reported in <10% of participants.

All participants reporting a TEAE in the Fast/Fed Cohort experienced Grade 1 (21.4%) or Grade 2 (14.3%) TEAEs. No participants reported Grade 3 or higher TEAEs.

There were no clinically significant differences in TEAEs reported among the participants treated with Compound A in the fasted and fed conditions.

PPI Cohort (n=17): The most common TEAEs (regardless of attribution to study drug) observed in ≥10% of participants were headache (5 participants, 29.4%) and COVID-19 (2 participants, 11.8%).

All participants reporting a TEAE in the PPI Cohort experienced Grade 1 (47.1%) or Grade 2 (5.9%) TEAEs. No participants reported Grade 3 or higher TEAEs.

There were no clinically significant differences in TEAEs reported among the participants treated with Compound A with PPI and participants treated with Compound A without PPI.

rBA Cohort (n=16): Only 1 participant (6.3%) reported a TEAE (dizziness, Grade 1).

There were no clinically significant differences in TEAEs reported among the participants treated with the 20% drug load and participants treated with the 42% drug load.

### Treatment Related Adverse Events (TRAEs)

A total of 3 of the total 47 participants had at least 1 TEAE considered potentially related to Compound A (regardless of severity grade).

Fast/Fed Cohort (n=14): 1 participant (7.1%) reported a TRAE of pollakiuria.

PPI Cohort (n=17): 2 participants (11.8%) reported at least 1 TRAE. TRAEs reported include nausea, headache, and pruritus (1 participant [5.9%] each).

rBA Cohort (n=16): No participants reported a TRAE.

### Serious Adverse Events (SAEs), Serious Adverse Drug Reactions (SARs) and Deaths

No SAEs, SARs or deaths were reported in the Clin Pharm Study (all cohorts).

### Treatment-Emergent Adverse Events (TEAEs) Leading to Discontinuation

One healthy participant reported a TEAE that led to discontinuation of the Clin Pharm Study. This participant was in the PPI cohort, treated with 200 mg Compound A and no PPI, and was unable to be dosed with Compound A for the second time, with PPI, due to COVID.

### Example 4: Safety and Efficacy in Patients with Breast Cancer (the FIH Study)

I. Safety in Patients with Breast Cancer (the FIH Study)

TEAEs and TRAEs from the clinical study database were coded to system organ class and preferred term using MedDRA version 22.0 and SAEs from the centralized safety database were coded using MedDRA version 25.0. The severity of adverse events was graded according to the NCI CTCAE version 5.0.

A TEAE is an AE occurring after the first dose of Compound A and within 30 days of the last Compound A dose regardless of study drug attribution or grade.

A TRAE is an AE assessed as “Possibly Related,” “Probably Related,” or “Related” to Compound A by the investigator.

Compound A Monotherapy—Part A—Dose Escalation (n=78 Patients):

### Treatment-Emergent Adverse Events (TEAEs)

Based on a 6 Jun. 2022 data cut-off, a total of 71 of the 78 patients (91.0%) in Part A (dose escalation) reported at least 1 TEAE.

The most common TEAEs (regardless of attribution to study drug) observed in ≥10% of patients across all Compound A doses, listed in decreasing order of frequency, were: fatigue, nausea, constipation, arthralgia, vomiting, back pain, diarrhea, headache, decreased appetite, aspartate aminotransferase (AST) increase, hot flush, hyperglycemia, pain in extremity, insomnia, alanine aminotransferase (ALT) increased, and dizziness (Table 6).

Most patients who experienced TEAEs reported Grade 1 (34.6%) or Grade 2 (39.7%) TEAEs. A total of 13 patients (16.7%) experienced a TEAE of Grade ≥3: 9 patients (11.5%) had Grade 3 TEAEs, 3 patients (3.8%) had Grade 4 TEAEs, and 1 patient (1.3%) had a Grade 5 TEAE.

The Grade 3 TEAEs that occurred in 1 patient each were nausea, stomatitis, arthralgia, fatigue, aspartate aminotransferase increased, electrocardiogram QT prolonged, amylase increased, blood alkaline phosphatase increased, lipase increased, headache, presyncope, embolism, embolism venous, procedural pain, humerus fracture, acute kidney injury, and malignant pleural effusion. Grade 3 hypertension occurred in 2 patients. The Grade 4 TEAEs of hypoglycemia, neutrophil count decreased, and neutropenia occurred in 1 patient each. The Grade 5 TEAE of cardiac arrest occurred in 1 patient and was unrelated to Compound A.

### Treatment Related Adverse Events (TRAEs)

A total of 54 of 78 patients (69.2%) had at least 1 TEAE considered potentially related to Compound A in Part A (dose escalation).

The most common TRAEs, observed in ≥10% patients, included nausea, fatigue, constipation, arthralgia, decreased appetite, hot flush, and vomiting (Table 6).

Most patients experienced TRAEs that were Grade 1 (42.3%) or Grade 2 (21.8%). Four patients (5.1%) experienced 6 Grade 3 TRAEs: headache lasting 1 day, asymptomatic amylase increased and lipase increased, nausea and asymptomatic electrocardiogram QT prolonged, and embolism venous that occurred after a minor procedure (bone biopsy). There were no Grade 4 or Grade 5 TRAEs reported in Part A.

TABLE 6
Treatment-Emergent All Causality AEs Reported in ≥ 10% and Treatment-Related AEs
Reported in ≥ 10% of Patients by Preferred Term Overall in Part A (the FIH Study,
All-treated Analysis Set)

| Preferred | Cpd A | Cpd A | Cpd A | Cpd A | Cpd A | Cpd A | 360 mg | Cpd A | Cpd A | |
|---|---|---|---|---|---|---|---|---|---|---|
| Term | 30 mg | 60 mg | 100 mg | 120 mg | 180 mg | 200 mg | Cpd A | 500 mg | 700 mg | Total |
| [n (%)] | (N = 3) | (N = 3) | (N = 9) | (N = 7) | (N = 7) | (N = 8) | (N = 15) | (N = 22) | (N = 4) | (N = 78) |
| Treatment- | | | | | | | | | | |
| Emergent | | | | | | | | | | |
| AEs | | | | | | | | | | |
| Fatigue | 0 | 1 | 2 | 0 | 2 | 1 | 4 | 14 | 2 | 26 |
| | | (33.3) | (22.2) | | (28.6) | (12.5) | (26.7) | (63.6) | (50.0) | (33.3) |
| Nausea | 0 | 2 | 2 | 2 | 2 | 3 | 4 | 9 | 2 | 26 |
| | | (66.7) | (22.2) | (28.6) | (28.6) | (37.5) | (26.7) | (40.9) | (50.0) | (33.3) |
| Constipation | 0 | 1 | 2 | 2 | 1 | 2 | 5 | 10 | 1 | 24 |
| | | (33.3) | (22.2) | (28.6) | (14.3) | (25.0) | (33.3) | (45.5) | (25.0) | (30.8) |
| Arthralgia | 0 | 1 | 2 | 2 | 2 | 1 | 2 | 4 | 0 | 14 |
| | | (33.3) | (22.2) | (28.6) | (28.6) | (12.5) | (13.3) | (18.2) | | (17.9) |
| Vomiting | 0 | 0 | 1 | 2 | 0 | 1 | 5 | 4 | 1 | 14 |
| | | | (11.1) | (28.6) | | (12.5) | (33.3) | (18.2) | (25.0) | (17.9) |
| Back pain | 0 | 0 | 1 | 1 | 0 | 1 | 2 | 6 | 2 | 13 |
| | | | (11.1) | (14.3) | | (12.5) | (13.3) | (27.3) | (50.0) | (16.7) |
| Diarrhea | 0 | 0 | 4 | 0 | 0 | 1 | 4 | 3 | 1 | 13 |
| | | | (44.4) | | | (12.5) | (26.7) | (13.6) | (25.0) | (16.7) |
| Headache | 0 | 1 | 1 | 2 | 0 | 1 | 4 | 3 | 1 | 13 |
| | | (33.3) | (11.1) | (28.6) | | (12.5) | (26.7) | (13.6) | (25.0) | (16.7) |
| Decreased | 0 | 0 | 2 | 3 | 0 | 0 | 4 | 2 | 1 | 12 |
| appetite | | | (22.2) | (42.9) | | | (26.7) | (9.1) | (25.0) | (15.4) |
| Aspartate | 0 | 0 | 0 | 3 | 2 | 3 | 0 | 1 | 2 | 11 |
| amino- | | | | (42.9) | (28.6) | (37.5) | | (4.5) | (50.0) | (14.1) |
| transferase | | | | | | | | | | |
| increased | | | | | | | | | | |
| Hot flush | 1 | 0 | 1 | 1 | 0 | 1 | 2 | 5 | 0 | 11 |
| | (33.3) | | (11.1) | (14.3) | | (12.5) | (13.3) | (22.7) | | (14.1) |
| Hyper- | 0 | 0 | 0 | 1 | 0 | 1 | 2 | 5 | 1 | 10 |
| glycemia | | | | (14.3) | | (12.5) | (13.3) | (22.7) | (25.0) | (12.8) |
| Pain in | 0 | 1 | 0 | 2 | 1 | 0 | 1 | 5 | 0 | 10 |
| extremity | | (33.3) | | (28.6) | (14.3) | | (6.7) | (22.7) | | (12.8) |
| Insomnia | 1 | 0 | 0 | 1 | 0 | 1 | 1 | 4 | 1 | 9 |
| | (33.3) | | | (14.3) | | (12.5) | (6.7) | (18.2) | (25.0) | (11.5) |
| Alanine | 0 | 0 | 0 | 1 | 2 | 2 | 1 | 2 | 0 | 8 |
| amino- | | | | (14.3) | (28.6) | (25.0) | (6.7) | (9.1) | | (10.3) |
| transferase | | | | | | | | | | |
| increased | | | | | | | | | | |
| Dizziness | 0 | 1 | 0 | 1 | 0 | 0 | 2 | 2 | 2 | 8 |
| | | (33.3) | | (14.3) | | | (13.3) | (9.1) | (50.0) | (10.3) |
| Treatment- | | | | | | | | | | |
| Related AEs | | | | | | | | | | |
| Nausea | 0 | 2 | 1 | 2 | 2 | 2 | 3 | 7 | 1 | 20 |
| | | (66.7) | (11.1) | (28.6) | (28.6) | (25.0) | (20.0) | (31.8) | (25.0) | (25.6) |
| Fatigue | 0 | 1 | 1 | 0 | 1 | 0 | 3 | 9 | 2 | 17 |
| | | (33.3) | (11.1) | | (14.3) | | (20.0) | (40.9) | (50.0) | (21.8) |
| Constipation | 0 | 0 | 1 | 1 | 0 | 2 | 3 | 3 | 0 | 10 |
| | | | (11.1) | (14.3) | | (25.0) | (20.0) | (13.6) | | (12.8) |
| Arthralgia | 0 | 1 | 1 | 2 | 1 | 1 | 0 | 2 | 0 | 8 |
| | | (33.3) | (11.1) | (28.6) | (14.3) | (12.5) | | (9.1) | | (10.3) |
| Decreased | 0 | 0 | 2 | 3 | 0 | 0 | 2 | 1 | 0 | 8 |
| appetite | | | (22.2) | (42.9) | | | (13.3) | (4.5) | | (10.3) |
| Hot flush | 0 | 0 | 1 | 0 | 0 | 1 | 1 | 5 | 0 | 8 |
| | | | (11.1) | | | (12.5) | (6.7) | (22.7) | | (10.3) |
| Vomiting | 0 | 0 | 0 | 2 | 0 | 1 | 2 | 3 | 0 | 8 |
| | | | | (28.6) | | (12.5) | (13.3) | (13.6) | | (10.3) |

n = number of patients;
Note:
For each preferred term, patients are counted only once.
Note:
AEs were coded using MedDRA version 22.0.

Compound A Monotherapy—Part B—Dose Expansion (n=71 Patients):

### Treatment-Emergent Adverse Events (TEAEs)

Based on a 6 Jun. 2022 data cut-off, a total of 62 of the 71 patients (87.3%) in Part B (dose expansion) reported at least 1 TEAE. The most commonly reported TEAEs (regardless of attribution to study drug) observed in ≥10% of patients, listed in decreasing order of frequency, include fatigue, nausea, constipation, arthralgia, aspartate aminotransferase increased, decreased appetite, headache, and back pain (Table 7).

Most patients who experienced TEAEs reported Grade 1 (29.6%) or Grade 2 (35.2%) TEAEs. A total of 16 patients (22.5%) experienced a total of 23 TEAEs of Grade ≥3 severity: 13 patients (18.3%) had Grade 3 TEAEs, 2 patients (2.8%) had Grade 4 TEAEs, and 1 patient (1.4%) had a Grade 5 TEAE.

The Grade 3 TEAEs that occurred in 1 patient each were intestinal obstruction, muscular weakness, aspartate aminotransferase increased, alanine aminotransferase increased, blood alkaline phosphatase increased, electrocardiogram QT prolonged, brain oedema, seizure, syncope, thrombocytopenia, cholangitis, neutropenia, spinal cord compression, upper gastrointestinal hemorrhage, fatigue, pyrexia, back pain, and decreased appetite. The Grade 3 TEAE of anemia was reported in 2 patients. The Grade 4 TEAEs of hyperbilirubinemia, hypercalcemia and hepatic hemorrhage were reported in 1 patient each. A Grade 5 TEAE of acute respiratory failure was reported in 1 patient, after that patient presented with progressive malignant pleural effusion.

### Treatment Related Adverse Events (TRAEs)

A total of 51 patients (71.8%) experienced at least 1 TEAE considered potentially related to Compound A (Table 7).

The most common TRAEs, observed in ≥10% of patients, include fatigue, nausea, and arthralgia (Table 7).

Most patients experienced TRAEs of Grade 1 (33.8%) or Grade 2 (31.0%). Five patients (7.0%) experienced 6 TRAEs with severity of Grade ≥3, including Grade 3 events of electrocardiogram QT prolonged, thrombocytopenia, neutropenia, decreased appetite, and fatigue. There was 1 Grade 4 TRAE of hyperbilirubinemia. There were no Grade 5 TRAEs reported.

TABLE 7
Treatment-Emergent All Causality AEs Reported in ≥10%
and Treatment-Related AEs Reported in ≥10% of
Patients by Preferred Term Overall in Part B
(the FIH Study, All-treated Analysis Set)

| | |
|---|---|
| | Total |
| Preferred Term [n (%)] | (N = 71) |

Treatment-Emergent AEs

| | | |
|---|---|---|
| Fatigue | 28 | (39.4) |
| Nausea | 15 | (21.1) |
| Arthralgia | 12 | (16.9) |
| Constipation | 12 | (16.9) |
| Aspartate aminotransferase increased | 9 | (12.7) |
| Back pain | 8 | (11.3) |
| Decreased appetite | 8 | (11.3) |
| Headache | 8 | (11.3) |

Treatment-Related AEs

| | | |
|---|---|---|
| Fatigue | 24 | (33.8) |
| Nausea | 12 | (16.9) |
| Arthralgia | 9 | (12.7) |

n = number of patients;
Note:
For each preferred term, patients are counted only once.
Note:
AEs were coded using MedDRA version 22.0.

Compound A Monotherapy—Part A—Dose Escalation (n=78 Patients):

Based on a 6 Jun. 2022 data cut-off, 8 of the 78 patients reported a total of 11 SAEs (Table 8). One SAE of venous embolism was assessed as possibly related and another SAE of embolism was assessed as unlikely related to Compound A by the investigator. All other SAEs reported in Part A were assessed as not related to Compound A. The Grade 3 venous embolism was considered possibly related to Compound A and reported as a SUSAR.

TABLE 8
Serious Adverse Events Reported in the FIH Study
Part A (Data Cut-off Date of 6 JUN. 2022)

| | | | | | Investigator- |
|---|---|---|---|---|---|
| | | | Onset | | Assessed |
| Identifier | Event | Dose | Day | Grade | Relatedness |
| Patient 1 | Procedural pain | 30 mg QD | 30 | 3 | Not related |
| Patient 2 | Humerus fracture | 250 mg BID | 59 | 3 | Not related |
| Patient 3 | Embolism venous^{a} | 360 mg QD | 42 | 3 | Possibly related |
| Patient 4 | Stomatitis | 200 mg QD | 79 | 3 | Not related |
| | Colitis | | | 1 | |
| | Neutrophil count | | | 4 | |
| | decreased | | | | |
| Patient 5 | Cardiac arrest | 250 mg BID | 26 | 5 | Not related |
| | Malignant pleural | | 10 | 3 | |
| | effusion | | | | |
| Patient 6 | Embolism | 360 mg QD | 54 | 3 | Unlikely related |
| Patient 7 | Hypoglycemia | 500 mg QD | 49 | 4 | Not related |
| Patient 8 | Acute kidney injury | 100 mg QD | 118 | 3 | Not related |

^{a}SAE reported as SUSAR.

Compound A Monotherapy—Part B—Dose Expansion (n=71 Patients):

Based on a 6 Jun. 2022 data cut-off, 9 of the 71 patients reported a total of 14 SAEs (Table 9). Two SAEs, hyperbilirubinemia and thrombocytopenia, experienced by 1 patient, were assessed by the investigator as possibly related to Compound A and were reported as SUSARs. All other SAEs reported in Part B were assessed as not related to Compound A.

TABLE 9
Serious Adverse Events Reported in the FIH Study
Part B (Data Cut-off Date of 6 JUN. 2022)

| | | | | Investigator- |
|---|---|---|---|---|
| | | Onset | | assessed |
| Identifier | Event | Day | Grade | Relatedness |
| Patient 1 | Seizure | 263 | 3 | Not related |
| Patient 2 | Intestinal obstruction | 85 | 3 | Not related |
| Patient 3 | Muscular weakness | 165 | 3 | Not related |
| | Back pain | 196 | 2 | |
| Patient 4 | Hepatic hemorrhage | 55 | 4 | Not related |
| Patient 5 | Hypercalcemia | 53 | 4 | Not related |
| Patient 6 | Hyperbilirubinemia ^{a} | 15 | 4 | Possibly related |
| | Thrombocytopenia ^{a} | | 3 | Possibly related |
| | Anemia | | 3 | Not related |
| | Cholangitis | | 3 | Not related |
| | Acute respiratory failure | 19 | 5 | Unlikely related |
| Patient 7 | Upper gastrointestinal | 36 | 3 | Not related |
| | hemorrhage | | | |
| Patient 8 | Spinal cord compression | 22 | 3 | Not related |
| Patient 9 | Pyrexia | 27 | 3 | Not related |

^{a} SAE reported as SUSAR.

II. Efficacy in Patients with Breast Cancer (the FIH Study)

Based on a 6 Jun. 2023 data cut-off, evidence of preliminary clinical activity was observed, as assessed by CBR and ORR, in the monotherapy Part A dose escalation cohort of the FIH Study.

Per protocol, the CBR evaluable analysis set included all treated patients who were enrolled at least 24 weeks prior to the data cut-off of analysis. CBR was defined as a confirmed CR or PR per RECIST v1.1 or SD with a minimum duration of 24 weeks.

In Part A, monotherapy dose escalation, the CBR was 36.1% (95% CI: 25.9%, 47.4%) in 30 of 83 evaluable patients.

Per protocol, the response-evaluable analysis set included all treated patients who had measurable disease at baseline and ORR was based on confirmed responses per RECIST v1.1.

In Part A, monotherapy dose escalation, the ORR was 11.5% (95% CI: 4.7%, 22.2%), with 7 of 61 response-evaluable patients having confirmed PR.

Clinical benefit by CBR was observed in CDK4/6 inhibitor-pretreated patients with ER+/HER2− BC in all Part A dose cohorts.

Efficacy data collection is ongoing for Part B.

### Example 5: Primary Endpoint—Clinical Benefit Rate (CBR)^{a}

The phase 2 expansion (VERITAC) of a phase 1/2 study tested 2 Compound A doses (200 mg once daily [QD] and 500 mg QD) in heavily pretreated patients (pts) with ER+/HER2-advanced breast cancer. Compound A 200 mg QD was selected as the phase 3 monotherapy dose based on comparable efficacy and favorable tolerability vs 500 mg QD and robust ER degradation (data cutoff: Jun. 6, 2022). Compound A 200 mg QD data is presented after 5 additional months of follow-up.

Methods: Compound A was administered to pts with ER+/HER2− locally advanced/metastatic breast cancer who had received ≥1 prior endocrine therapy for ≥6 months, ≥1 cyclin-dependent kinase (CDK)4/6 inhibitor, and ≤1 chemotherapy regimen. The primary endpoint was clinical benefit rate (CBR; rate of confirmed complete or partial response or stable disease ≥24 weeks).

Results: As of Nov. 1, 2022, 35 patients (median age: 63 y [range: 42-79]; 97% female) received Compound A 200 mg QD. Pts had a median of 4 prior regimens (range: 1-9); 100% prior CDK4/6 inhibitors, 74% prior fulvestrant, and 74% prior chemotherapy (46% in metastatic setting). CBR with Compound A 200 mg QD was 37.1% (95% CI: 21-55) in all evaluable pts (n=35) and 47.4% (95% CI: 24-71) in those with mutant ESRI (n=19). CBR was 40.0% (95% CI: 19-64) and 33.3% (95% CI: 12-62) in evaluable pts with WT (n=20) and mutant PIK3CA (n=15), respectively. Median progression-free survival in all evaluable pts was 3.5 months (95% CI: 1.8-7.8). As of the data cutoff date, 14 pts were on treatment for ≥24 weeks (4 for ≥48 weeks) with 4 ongoing. Substantial on-treatment reductions in mutant ESRI circulating tumor DNA levels were observed. Treatment-related adverse events in ≥10% of pts were fatigue (40%), hot flush (17%), nausea (14%), arthralgia (11%), and increased aspartate aminotransferase (11%); all were grade 1/2.

TABLE 10
CBR Summary

| | 200 mg QD | 500 mg QD | Total |
|---|---|---|---|
| | (n = 35) | (n = 36) | (N = 71) |
| CBR, % (95% CI) | 37.1 (21.5- | 38.9 (23.1- | 38.0 (26.8- |
| | 55.1) | 56.5) | 50.3) |
| Patients with | (n = 19) | (n = 22) | (n = 41) |
| mutant ESR1 | | | |
| CBR, % (95% CI) | 47.4 (24.4- | 54.5 (32.2- | 51.2(35.1- |
| | 71.1) | 75.6) | 67.1) |

Based on a 6 Jun. 2022 data cut-off, preliminary efficacy was seen in the Part B dose expansion (VERTIAC) of this study. The CBR in the overall population was 37.1% (95% CI: 21.5%, 55.1%) and 38.9% (95% CI: 23.1%, 56.5%) at 200 mg and at 500 mg respectively.

The CBR in evaluable patients with estrogen receptor 1 gene (ESR1) mutations, was similar between the doses: CBR was 47.4% in patients at 200 mg QD (95% CI: 24.4%, 71.1%) and 54.5% at 500 mg QD (95% CI: 32.2%, 75.6%).
CBR consistent with Phase 1 dose escalation data
   Phase 1: 40% in all patients, 50% in patients with ESR1-mutant tumors
Patients with WT ESRI (n=25) exhibited CBR rate of 20%

### Phase 3 2L+ Monotherapy Trial for Compound A

### Trial Will:

Include prior CDK4/6; and

Exclude patients with prior fulvestrant or prior chemotherapy in the metastatic setting.

Eight patients in the Phase 2 expansion cohort for Compound A did not have prior fulvestrant or prior chemotherapy in the metastatic setting (consistent with the Phase 3 trial design). The CBR was 62.5% (5 of 8) in these patients versus 38% (27 of 71) in the intent to treat (ITT) population. Notably, three of the eight patients discontinued treatment in November 2022*; the continuing on therapy had durations of eight to fourteen months.

Summary: After longer follow-up, Compound A 200 mg QD continued to show clinical activity and was well tolerated in heavily pretreated pts with ER+/HER2− advanced breast cancer. The ongoing phase 3 VERITAC-2 study is evaluating Compound A 200 mg QD versus fulvestrant.

TABLE 11
Summary Monotherapy Trial for Compound A.

| | | | | Prior | | |
|---|---|---|---|---|---|---|
| | | | | Chemo- | | |
| | | | | therapy | | |
| | | Prior | | in a | | |
| | | CDK4/6 | Prior | metastatic | | |
| | | Inhibitor | Fulvestrant | setting | CBR | PFS |
| ITT | (n = | 100% | 78.9% | 45.1% | 38.0% | 3.7 |
| | 71) | | | | | months |
| ITT | (n = | | | | 51.2% | 5.7 |
| ESR1m | 41) | | | | | months |
| No prior | (n = | 100% | 0% | 0% | 62.5% | NR* |
| | 8)^{‡} | | | | | |

^{‡}All patients in subset had ESRI mutations;
*median PFS had not been reached by November 2022.

### CONCLUSION

Without wishing to be bound by theory, based on the foregoing Examples, a 200 mg daily dose may be beneficial for Compound A as a monotherapy.

### Example 6: Summary of Secondary Pharmacodynamics

In vitro studies revealed that Compound A bound to several other nuclear hormone receptors (AR, GR, progesterone receptor, and ERP) and the opioid kappa receptor. Further analysis indicated that ERP, but not AR or GR, was degraded by Compound A. ERβ and ERα have homology in their ligand-binding domains and many potent ER ligands display binding to both receptors, including currently approved drugs tamoxifen and raloxifene.

Compound A engages the E3 ligase cereblon at the same binding site as IMiDs, such as thalidomide, lenalidomide, and pomalidomide. These drugs bind to cereblon and promote the degradation of novel (neomorphic) substrates. In vitro studies demonstrated that Compound A did not degrade the known cereblon neomorphic substrates Ikaros, Aiolos, GSPT1, or CK1α. Compound A displayed some degradation activity against the cereblon neomorphic substrate SALL4 in one cell line, but no SALL4 degradation in a different cell line. While the teratogenic effects of the thalidomide class have been linked to their SALL4-degradation activity, no significant adverse effects are expected with partial loss of SALL4 as most adult tissues do not express SALL4.

### Example 7: Summary of Safety Pharmacology

The potential for Compound A and Compound B to impact vital functions was assessed in vitro and in single- and repeat-dose in vivo studies.

Neither Compound A nor Compound B directly inhibit the hERG channel with measured IC₅₀ of >25.1 μM and >20 μM, respectively. Based on the recommended Phase 3 dose of 200 mg, the unbound Cₘₐₓ margins are approximately 170× and 499× for Compound A and Compound B, respectively.

A lead-in single-dose TK and tolerability study conducted prior to the telemetry study indicated approximately dose-proportional increase in exposure between 200 and 400 mg/kg in the dog. In a dedicated GLP cardiovascular assessment, a single dose of Compound A was administered via oral gavage to telemetered conscious male and female beagle dogs at doses of 0, 90, 200, and 400 mg/kg in a modified Latin-square design. Plasma samples for determination of Compound A and Compound B concentrations were obtained predose and approximately 4 hours postdose during the cardiovascular phase. Following completion of the cardiovascular phase, all animals were administered a single oral dose of Compound A at 400 mg/kg for assessment of TK parameters. No Compound A-related effects were noted after oral administration of Compound A at 90 and 200 mg/kg. Following administration of Compound A at 400 mg/kg, a statistically significant 7 ms shortening of the PRinterval was noted from 1 to 3.25 hours postdose. There were no changes in QT- or QTc-interval and no other Compound A related effects were noted at 400 mg/kg. At 400 mg/kg (the only dose studied in the TK phase), dogs (n=8, males and females combined) achieved a mean of 16,000 ng/mL for Cₘₐₓ, 5.1 hours for Tₘₐₓ, and 272,000 ng·h/mL for AUC₀₋₂₄.

In the 1-month GLP toxicology dog study, oral administration of Compound A did not elicit any changes in ECG evaluations after 28 days of dosing up to the highest tested dose of 90 mg/kg/day. No changes were noted on PR-interval, QRS duration, QT or QTc intervals, or heart rate, and there were no arrhythmias or abnormal waveforms observed. In the 3-month GLP study, oral administration of Compound A at 90 mg/kg/day produced nonadverse increases in QT-, QTc-, and PR-intervals on Day 85. There were no arrhythmias or abnormal waveforms observed.

Related to respiratory and CNS safety pharmacology, there was no effect of Compound A on respiratory-related clinical signs, CNS function and body temperature up to the highest test dose of 100 mg/kg/day in rats, as shown in FOB testing in the 28-day GLP study. Additionally, no clinical signs or microscopic changes suggestive of alterations in respiratory or CNS function were noted in the 3-month toxicity rat study. Additionally, with regards to potential effects in the CNS, Compound A does not readily cross the blood brain barrier and there is limited CNS exposure.

### Example 8: Summary of Pharmacokinetics and Product Metabolism in Animals

The PK of Compound A was characterized following a single PO or intravenous dose of Compound A in the mouse, rat, dog, and monkey and following QD PO dose for 5 days in the mouse, rat, and dog. The interconversion between Compound A and Compound B, the epimer with ER antagonistic but no degradation activity, was characterized in the mouse, rat, dog, and monkey following PO dosing of either Compound A or Compound B. The TK profiles of both Compound A and Compound B were assessed in the rat and dog toxicology studies. In vitro studies were conducted to characterize permeability, metabolic stability in plasma and liver fractions, plasma protein binding, blood cell partitioning, and metabolite profiles. In vitro studies to assess potential for DDIs included the identification of the predominant isoforms of CYP involved in Compound A metabolism, inhibition, and induction potentials against major human CYP isoforms, and Compound A involvement as a substrate of and inhibition against selected human drug transporters. Because the patient population for this indication is primarily female, most of these studies were conducted in female animals and cellular/subcellular fractions; however, the data also support inclusion of male patients.

### Methods of Analysis

Non-chiral LC-MS/MS methods were used to analyze Compound A from multiple ADME studies. Chiral LC-MS/MS methods were used to analyze both Compound A and Compound B in selected animal PK studies and validated in rat and dog plasma over a concentration range of 1.00 to 1000 ng/mL to analyze both Compound A and Compound B in support of the GLP toxicity studies in these species.

### Pharmacokinetics

The PK profile of Compound A in the preclinical species (mouse, rat, dog, and monkey) was characterized by a low to moderate clearance (13.8% to 33.8% of hepatic blood flow), extensive tissue distribution (2.1 to 6.0 L/kg), short to moderate t_{1/2} (2.1 to 8.2 hours), and moderate to good oral bioavailability (27% to 65%).

Dose-dependent increase in Compound A exposure was observed when Compound A was administered as an oral solution in the mouse (10, 30, and 100 mg/kg), rat (30, 100, and 300 mg/kg), dog (15, 45, 90, 200, and 400 mg/kg), and monkey (1 and 3 mg/kg).

The performance of the Compound A 50 mg tablet used in the FIH study was evaluated in 2 different groups of 6 female dogs under fed and fasted conditions. For 6 days, dogs received a single 50 mg tablet QD after pre-treatment with pentagastrin. Little accumulation was observed over the course of the study. In addition, exposure in chow-fed dogs was similar to that obtained for solution formulations administered at a similar dose. To reduce the pill burden, a 100 mg tablet was developed and when tested in dogs, similar exposure was observed when dogs were dosing with a 50 mg tablet.

A 3-fold increase in AUC and reduced inter-animal variability was observed in fed dogs, and as such, the data indicated that the tablets should be administered with food in clinical trials.

The extent of epimerization was limited (<15%) in the mouse, rat, and dog and negligible in the monkey when dosed with either Compound A or Compound B. Additionally, the limited exposure of Compound B relative to Compound A (≤26%) was confirmed in toxicology studies in both the rat and dog receiving daily administration of Compound A.

### Absorption

In an in vitro bidirectional permeability study in Caco-2 cells, Compound A at 1.125 pM showed low permeability of 1.33×10⁻⁶ cm/s in the apical to basolateral direction and 0.08×10⁻⁶ cm/s in the basolateral to apical direction. Similarly low bidirectional permeability was obtained at 11.25 μM.

Taken together, the data suggest that Compound A is unlikely to be a substrate of intestinal efflux transporters including P-gp and BCRP, which was further confirmed in an inside-out vesicular assay format.

Moderate to good oral bioavailability was generally obtained when Compound A was dosed as a solution in preclinical species, despite its low solubility (1.5 μg/mL in phosphate buffer at pH 7.4) and low permeability.

Studies in fasted rats and dogs conducted during Compound A spray-dried dispersion formulation development showed a pH effect on absorption when administered with famotidine (lowered PK at neutral pH). However, these data do not characterize the fed conditions used in the clinical setting, as PPIs may have the potential to affect the absorption of Compound A even with food.

### Distribution

Compound A plasma protein binding was moderately high across species, with the free fraction averaging 6.10% in the mouse, 8.2% in the rat, 2.6% in the dog, 4.0% in the monkey, and 12.4% in human at 1 μM. The blood cell partitioning of Compound A in human blood at 5 pM was limited, with a blood cell/plasma concentration ratio of 0.18.

Consistent with the large V_{SS} in the rat (6 L/kg), the tissue distribution of Compound A was extensive in the rat receiving QD oral dose of Compound A at 100 mg/kg/day for 3 days. The tissue plasma ratio ranged from ≤0.1 (brain) to 5.6 to 13.9 (lung). Tissue distribution data showed no signs of tissue accumulation upon repeat-dose.

### Metabolism

Compound A demonstrated relatively low turnover in liver microsomes and hepatocytes across species. Several metabolic pathways, including oxidation, dehydrogenation, dealkylation, sulfation, and glucuronidation, and a combination of oxidation and dealkylation, reduction, or methylation, were evident in liver microsomes and hepatocytes, along with hydrolysis in plasma, suggesting the involvement of both CYP-mediated oxidative and non-CYP-mediated metabolism in Compound A clearance. No human unique metabolites were observed. Although it is not thought to be formed via enzymatic conversion, the epimer Compound B was formed to a limited extent in animals.

### Example 9: Summary of Toxicology

Nonclinical toxicology studies were conducted with Compound A to evaluate the potential toxicity and determine the TK profile of Compound A and its epimer Compound B when Compound A was administered QD orally (by gavage). The toxicology program includes up to 3-month GLP-compliant repeat-dose studies in rats and dogs, GLP-compliant in vitro bacterial reverse mutation (Ames) assays, in vitro and in vivo micronucleus assays, and a GLP-compliant in vitro 3T3 phototoxicity study. Both rats and dogs have the target (ER) for Compound A and have been shown to be sensitive in toxicology programs for agents that inhibit ER signaling (e.g., fulvestrant). Both species show a dose-dependent exposure following oral dosing of Compound A, and both show concordance of metabolic pathways in vitro compared to humans. The formulation of Compound A used in the toxicology studies was an oral solution in a vehicle of 63% (v/v) PEG 400, 27% (v/v) Vitamin E TPGS, and 10% (v/v) DMSO.

The pivotal nonclinical safety studies were conducted in the US in accordance with GLP regulations per the US 21 CFR Part 58. These include the cardiovascular safety pharmacology study in dogs, the pivotal 28-day and 3-month repeat-dose toxicology studies in rats and dogs, and an in vitro phototoxicity assay. The bacterial reverse mutation assay, in vitro micronucleus assay, and 2 in vitro hERG studies were conducted in the UK in accordance with the UK Good Laboratory Practice Monitoring Authority, MHRA: Good Laboratory Practice Regulations 1999, Statutory Instrument 1999 No. 3106 as amended by the Good Laboratory Practice (Codification Amendments Etc.) Regulations, 2004, Statutory Instrument 2004, No. 994; and the OECD GLP and OECD Principles on Good Laboratory Practice ENV/MC/CHEM (98) 17 (Revised in 1997, Issued January 1998).accordance with the OECD Principles of Good Laboratory Practice and as accepted by Regulatory Authorities throughout the European Union, United States of America (FDA), Japan (MHLW), and other countries that are signatories to the OECD Mutual Acceptance of Data Agreement.

### Single-Dose Studies

Compound A administered by a single oral gavage dose to beagle dogs at doses of 200 mg/kg or 400 mg/kg was tolerated with clinical observations limited to fecal changes at both doses and emesis at 400 mg/kg. Mean Cₘₐₓ and AUC₂₄ were 25,000 ng/mL (sexes combined) and 349,000 ng·h/mL (sexes combined), respectively, at 400 mg/kg the highest dose of Compound A evaluated by oral administration.

### Repeat-Dose Studies

### 7-Day Oral Dose Range Finding Toxicity Study in Rats

Compound A was well tolerated following once-daily oral (gavage) administration to rats at dose levels up to 300 mg/kg/day for 7 days. Noteworthy findings were changes in the female reproductive organs, including in the ovaries (minimal to moderate increased size of tertiary follicles, retained corpora lutea, and minimal to moderate interstitial cell hypertrophy), uterus (atrophy) and vagina (mucosa atrophy). These findings were observed in all groups of treated females and were consistent with the pharmacologic activity of Compound A.

### 28-Day Oral Toxicity Study in Rats

Compound A was well tolerated following daily oral (gavage) administration of 0, 3, 10, 30, or 100 mg/kg/day to rats in the GLP-compliant 28-day toxicity study. There were no mortalities and no Compound A-related clinical observations or changes in BWs or food consumption, and no FOB observations or ophthalmology findings. Minor clinical pathology changes at the 100 mg/kg/day dose level (minimally higher lymphocyte count in males, minimally lower calcium concentration in both sexes, minimally lower albumin concentration in males, and mildly lower cholesterol concentration in females) were potentially Compound A-related but were reversible and were not considered adverse.

Compound A-related microscopic findings included increased ovarian cysts in females administered ≥3 mg/kg/day and atrophy of the uterus and of the cervical and vaginal mucosal epithelium in females administered ≥10 mg/kg/day. The microscopic findings in the female reproductive organs were reversible and considered to be due to the pharmacologic activity of Compound A. The microscopic findings of increased incidence (compared to controls) of minimal hemorrhage in the AV valves of the heart in males (≥3 mg/kg/day) and females (10 or 30 mg/kg/day) were without dose or plasma exposure relationship and were not associated with changes to the valve tissues, and no other myocardial tissue changes were observed. Similar findings had been noted in a few animals in the 7-day study. Notably, however, a subsequent 3 month study in rats showed there were no findings in the heart valves, suggesting these were spurious findings in the 28-day study.

The NOAEL for daily oral administration of Compound A to rats for 28 days was determined to be 100 mg/kg/day.

### 3-Month Oral Toxicity Study in Rats

Compound A was well tolerated following once daily oral (gavage) dosing in the GLP-compliant 3-month toxicity study in rats. Male and female rats, 10/sex/group, were administered 0 (vehicle), 30, 100, or 300 mg/kg/day. Assessment of toxicity was based on mortality, clinical observations, BWs, food consumption, ophthalmic examinations, and clinical and anatomic pathology. Blood samples for TK were obtained from a satellite group of animals.

There were no mortalities, treatment-related effects on food consumption, or urinalysis and there were no ophthalmic observations of note.

Clinical observations were limited to intermittent incidences of abnormal skin color (red, around the right or both eyes) observed in 1 control male, 1 female at 100 mg/kg/day, and 2 females at 300 mg/kg/day on a few occasions; this finding was considered nonadverse. The only other clinical observations were brief and transient whole-body convulsions noted in 2 animals at 100 mg/kg/day (1 male, 1 female) on single occasions and in 2 animals at 300 mg/kg/day (2 females) on a few occasions. Taking into consideration the timing of the convulsions relative to the dosing procedure (all were prior to, or shortly after dosing, prior to any significant systemic absorption, except the single incidence in 1 male at 100 mg/kg/day) and published reports of convulsions in this strain of rats due to oral gavage procedures the observations were considered nonadverse and are considered not related to Compound A.

Compound A caused changes in BW in treated animals, with males at 300 mg/kg/day showing slight decreases whereas females at 100 and 300 mg/kg/day showed slight increases.

Hematology findings included minimally higher WBC count and mildly increased lymphocyte count in females at 300 mg/kg/day, which correlated with the minimal to slight infiltrates of vacuolated macrophages in the lungs. No mechanism could be identified for the observed minimally higher red cell mass in all groups of treated females and prolonged prothrombin time was seen in both sexes at doses ≥100 mg/kg/day. Clinical chemistry changes were only observed at 300 mg/kg/day: minimally lower cholesterol (males), calcium (females), and glucose (males and females).

Compound A caused dose-related findings in the female reproductive organs. These included minimal to marked follicular cysts, minimal to slight follicular hemorrhage, and minimally to slightly smaller corpora lutea in the ovaries in females administered ≥30 mg/kg/day, correlating with macroscopic observations of cysts and large ovaries and increased ovary weights. Minimal to moderate uterine atrophy, and minimal to slight atrophy of the cervical and vaginal mucosal epithelium were observed in females administered ≥30 mg/kg/day; the uterine atrophy correlated with the observed decreased uterus/cervix weights. Findings in the ovaries and uterus were considered adverse due to the likely effect on fertility. Overall, the microscopic findings in the female reproductive organs were considered due to the pharmacologic activity of Compound A.

In males, decreases in prostate and epididymis organ weights were attributed to the pharmacologic activity of Compound A, but these lacked microscopic correlates.

Compound A dosing was associated with minimal (males) or minimal to slight (females) diffuse atrophy of the zona fasciculata in the adrenal cortex, which correlated with decreased adrenal gland weights in females. The adrenal findings were considered not adverse and likely to be adaptive changes.

The only other microscopic finding considered related to Compound A dosing was an increased incidence and severity (from minimal to slight) of infiltrates of vacuolated macrophages in the lungs of animals at 300 mg/kg/day; this finding was considered not adverse.

Organ weight changes observed but lacking in microscopic correlates included decreased kidney weights in males (300 mg/kg/day), minimally increased absolute liver weight or liver-to-body-weight ratio in both sexes, and decreased pituitary weights in males (300 mg/kg/day) and females (all dose levels).

TK analysis showed that sex differences in Cₘₐₓ and AUC₀₋₂₄ were less than 2-fold by the end of the study. Exposure increases with increasing dose level from 30 to 300 mg/kg/day in a generally dose-proportional manner in females but was greater than dose-proportional in males. No accumulation of Compound A was observed.

In summary, daily dosing up to 300 mg/kg/day in rats was tolerated with the NOAEL for 3 months of oral dosing in males determined to be the highest dose tested of 300 mg/kg/day. This dose level in males corresponded to Cₘₐₓ and AUC₀₋₂₄ values of 4,330 ng/mL and 88,500 ng·hr/mL, respectively, on Day 91. In females, a NOAEL was not determined based on pharmacologically driven adverse findings in the female reproductive tract. However, the lack of severe clinical signs, no overall impact on the health and wellbeing of the animals and the tolerability profile for females at 300 mg/kg/day are supportive of the continued evaluation of 300 mg/kg/day dose. This dose level in females corresponded to Compound A Cₘₐₓ and AUC₀₋₂₄ values of 3,710 ng/mL and 75,000 ng·hr/mL, respectively, on Day 91.

### 7-Day Oral Dose Range Finding Toxicity Study in Dogs

Compound A was well tolerated following once-daily oral (gavage) administration to dogs at dose levels up to 120 mg/kg/day for 7 days. Noteworthy findings were changes in the female reproductive organs including in the ovaries (increased size of tertiary follicles, granulosa cell hypertrophy/hyperplasia, theca cell hypertrophy/hyperplasia, and/or interstitial cell hypertrophy, retained corpus luteum), together with atrophy in the uterus, vaginal mucosa, and oviducts. These findings were observed in all groups of treated females and were consistent with the pharmacologic activity of Compound A. Decreased testis and epididymis weights in males were also attributed to the expected pharmacologic activity of Compound A.

### 28-Day Oral Toxicity Study in Dogs

Compound A was well-tolerated following daily oral (gavage) administration of 0, 15, 45, or 90 mg/kg/day to dogs in the GLP-compliant 28-day toxicity study. There were no mortalities and no Compound A-related clinical observations, changes in BW or food consumption or ophthalmic observations. There were no ARV-471-related ECG abnormalities observed, including no effects on PR-interval, QRS duration, QT or QTc intervals, or heart rate and no arrhythmias or abnormal waveforms. Clinical pathology findings considered related to Compound A were limited to minimally decreased cholesterol concentration in animals administered 90 mg/kg/day, which exhibited evidence of reversibility, lacked microscopic correlates, and was of unclear mechanism, and thus was considered nonadverse. Minimally to mildly increased glutamyl transferase activity in animals administered 90 mg/kg/day was of uncertain relationship to Compound A.

The main Compound A-related microscopic findings were in the female and male reproductive tissues and were consistent with the pharmacological effect of Compound A. In females, changes were observed in the ovary, including slightly to markedly cystic follicles and corpora lutea, correlating with observations of increased ovary weights, large ovaries among treated females in all dose groups, and bilateral fluid-filled cysts in 2 females at 90 mg/kg/day.

Findings were also observed in the uterus, including minimal to moderate atrophy at all dose levels, without dose-response. Following 28 days of recovery, the ovaries exhibited synchronization of activity with other reproductive tissues (considering the multiple corpora lutea, the minimal vaginal mucosal hypertrophy changes, and the reversal of uterine atrophy).

In male reproductive tissues, changes were seen in the testis (dose-dependent minimal to moderate interstitial [Leydig] cell hypertrophy, and minimal seminiferous tubule degeneration in all groups of treated males), epididymis (minimal to slight intraluminal cellular debris in all groups of treated males, likely correlated with increased epididymis weights), and prostate (minimal to slight hypertrophy in all groups of treated males correlated with increased prostate weights). Changes in the testis were still observed at the end of the recovery period, which may have reflected the relatively short withdrawal period (28 days) after degradation of the ER, compared to the time needed for recovery of ER levels followed by response of the tissues, including completion of the spermatogenic process for dogs (approximately 62 days).

Other microscopic changes attributed to Compound A were in the kidney of males administered ≥45 mg/kg/day (reversible, minimal to slight tubule degeneration/regeneration, likely correlated with increased kidney weights, and reversible minimal glomerular capsule fibrosis), adrenal cortex (minimal hypertrophy in females at 90 mg/kg/day) and thymus (minimally to slightly decreased lymphocytes in males at 90 mg/kg/day, correlating with macroscopic observations of small thymus and reduced thymus weights). The adrenal cortex and thymus changes were considered due to stress, secondary to Compound A dosing.

Overall, effects for the 90 mg/kg/day dose were considered nonadverse due to the mild severity of findings and the lack of impact on the health and well-being of animals, and acknowledging the pharmacological effects of the drug, the NOAEL for daily oral administration of Compound A to dogs for 28 days was determined to be 90 mg/kg/day.

### 3-Month Oral Toxicity Study in Dogs

Compound A was well tolerated following once-daily oral (gavage) dosing in the GLP-compliant 3-month toxicity study in dogs. Male and female Beagle dogs, 4/sex/group, were administered 0 (vehicle), 10, 30, or 90 mg/kg/day. Assessment of toxicity was based on clinical observations, BWs, food consumption, ophthalmology, ECG evaluations, and clinical and anatomic pathology.

There was no mortality and no Compound A-related findings in clinical signs, BWs, food consumption, and ophthalmology examinations.

Compound A produced increases in pre-dose and post-dose QT- and QTc-intervals and post-dose PR-intervals on Day 85 at the 90 mg/kg/day dose level when compared to baseline. These were considered nonadverse based on a lack of association with any other endpoints (arrhythmia, clinical signs, and microscopic findings in the heart).

Clinical pathology evaluations (hematology, coagulation, urinalysis) showed no changes, except for Compound A-related nonadverse clinical chemistry changes that included sporadic increases in ALT in males administered 10 or 30 mg/kg/day and a female administered 90 mg/kg/day, as well as increases in GGT in females administered 90 mg/kg/day and an increase in ALP in one male administered 90 mg/kg/day. Additional findings included decreases in cholesterol in males administered ≥30 mg/kg/day and sporadic increases in phosphorus in males administered ≥30 mg/kg/day and a female administered 90 mg/kg/day.

Compound A produced changes in female and male reproductive tissues, consistent with pharmacological effects of the drug. In females, there were findings in the ovary (increased number of corpus luteum, atrophy, and cysts; associated with enlargement observed macroscopically and higher ovarian weights), uterus (atrophy), cervix (atrophy), vagina (atrophy) and mammary gland (atrophy) at ≥10 mg/kg/day and in the oviduct (atrophy) at 90 mg/kg/day; these findings were considered adverse. In males, findings in the interstitial cells of the testis (hypertrophy) and prostate gland (hypertrophy; associated with higher prostate weights) at ≥10 mg/kg/day were considered nonadverse.

Additional, nonadverse findings attributed to Compound A were noted in the pituitary gland (hypertrophy of basophilic cells) at ≥30 mg/kg/day in males and 90 mg/kg/day in females.

Based on the Day 91 assessment of plasma TK, mean systemic exposure of Compound A increased with increasing dose in an approximately dose proportional manner on Day 91.

In summary, based on the findings in this toxicity study, 90 mg/kg/day was identified as the NOAEL for daily dosing for 3 months in males. A NOAEL was not identified for females based upon microscopic findings in the ovary, uterus, cervix, vagina, oviduct, and mammary gland at ≥10 mg/kg/day that were considered adverse, but it is acknowledged that these effects were consistent with the pharmacological effects of Compound A and were also observed in the 7- and 28-day studies in dogs. In males at 90 mg/kg/day, the Cₘₐₓ was 4,880 ng/mL and AUC₀₋₂₄ was 83,500 ng·h/mL on Day 91. At the high dose (90 mg/kg/day) for females, the associated Cₘₐₓ was 4,250 ng/mL, and AUC₀₋₂₄ was 73,100 ng·h/mL on Day 91.

### Genotoxicity Studies

Compound A was assayed for potential to cause mutations in a GLP-compliant bacterial reverse mutation study. Compound A did not induce mutation in 4 histidine-requiring strains (TA98, TA100, TA1535, and TA1537) of Salmonella typhimurium, and one tryptophan-requiring strain (WP2 uvrA) of Escherichia coli when tested under treatments at concentrations up to 5000 pg/plate, in the absence and in the presence of a rat liver metabolic activation system (Aroclor 1254-induced rat liver S9 fraction).

Compound A was tested in a GLP-compliant in vitro micronucleus assay. Compound A did not induce biologically relevant increases in the frequency of micronuclei in cultured human peripheral blood lymphocytes when tested up to precipitating concentrations for 3+21 hours in the absence and presence of a rat liver metabolic activation system (S9) and when tested up to the limit of toxicity for 24+24 hours in the absence of S9.

Following a toleration lead-in evaluation, Compound A administered at doses of 0, 400, 800 and 1600 mg/kg/day for two consecutive days did not induce chromosomal damage in reticulocytes in the rat micronucleus test.

Maternal and embryo-fetal effects of Compound A were evaluated in presumed pregnant female Sprague-Dawley rats in a preliminary embryo-fetal development study. Time-mated and confirmed pregnant female rats (n=8) received control article or 30, 100, or 300 mg/kg/day Compound A via oral gavage once daily from Gestation Days (GD) 6 through GD 17. Toxicokinetics were assessed in satellite animals. Based on the draft report there was no mortality in the study with Compound A-related clinical signs noted as red vaginal discharge at doses ≥30 mg/kg/day, indicative of pregnancy loss. Decreases in maternal body weight and maternal body weight gain noted during the gestation phase (pregnant animals only) were considered secondary to Compound A-related higher post-implantation loss noted at ≥30 mg/kg/day. Mean maternal body weights at 100 mg/kg/day (4 pregnant animals) were lower than the control group mean on GDs 15, 18, and 21 (75% to 92% of controls).

At planned necropsy (GD 21), pregnancy was confirmed in 7/8, 7/8, 4/8, and 0/8 females in the control, 30, 100, and 300 mg/kg groups, respectively. For the TK animals (necropsy on GD 18), there were 3/3, 5/6, 3/6, and 0/6 female rats that were confirmed pregnant in the control, 30, 100, and 300 mg/kg groups, respectively. With initiation of dosing of Compound A on GD 6, an estrogen-dependent time period, it is possible that loss of pregnancy occurred shortly after the start of dosing. As a result of possible early loss of pregnancy, there were no readily identifiable implantation sites at microscopic evaluations in 100% of the female rats at 300 mg/kg/day and 50% of the rats at 100 mg/kg/day. Compound A related effects on embryo-fetal viability and growth at ≥30 mg/kg/day included higher post implantation loss (47.31% and 100%, respectively) due to an increased in early resorptions; lower mean number of live fetuses (7.0 and no live fetuses/litter, respectively, compared with 11.3 live fetuses/litter in controls) at 30 and 100 mg/kg/day, and lower mean fetal body weights at 30 mg/kg/day. Maternal administration of Compound A did not produce any external or visceral malformations or variations in the surviving fetuses at 30 mg/kg/day. Based on the limited number of fetuses evaluated, there was a low incidence of skeletal abnormalities at 30 mg/kg/day. There were delays in skeletal ossification, based on lower mean numbers of ossified caudal vertebrae, hindlimb metatarsals, and hindlimb phalanges, at 30 mg/kg/day that were consistent with lower mean fetal body weights in the surviving fetuses in this dose group.

The developmental NOAEL for Compound A could not be established based on lower fetal body weights at ≥30 mg/kg/day and lower embryo-fetal viability at ≥30 mg/kg/day. The 30 mg/kg/day dose level corresponded to a Cₘₐₓ of 291 ng/mL and AUCₗₐₛₜ of 4,380 ng·h/mL on GD 17. The effects observed in this study are consistent with the primary pharmacology of Compound A and the well-established biological importance of estrogen signaling in pregnancy.

### Phototoxicity Studies

Since UV-vis absorption spectrum of Compound A showed absorbance above 290 nm, an in vitro study was conducted to evaluate the phototoxic potential of Compound A as measured by the relative reduction in viability of BALB/c 3T3 mouse fibroblasts exposed to Compound A in the absence and presence of ultraviolet radiation (20325699). When tested up to its limit of solubility in 1% DMSO/DPBS (10.0 μg/mL), Compound A did not display an IC₅₀ either with or without UVR exposure. Accordingly, results from this in vitro study indicate that Compound A is not phototoxic.

### Example 10—Updated Safety and Efficacy Findings in Patients with Breast Cancer

Overall TEAEs were observed in 143 of the total 154 patients (92.9%) treated with Compound A in the ongoing ARV—471-mBC-101 study (Parts A and B), as of 6 Jun. 2023.

### Treatment-Emergent AEs (TEAEs)

As of 6 Jun. 2023, a total of 80 of the 83 patients (96.4%) in Part A (dose escalation) reported at least 1 TEAE.

The most common TEAEs of all grades (regardless of attribution to study drug) observed in ≥10% of patients across all Compound A doses listed in decreasing order of frequency were fatigue, constipation, nausea, arthralgia and headache (Table 12).

Most reported TEAEs are Grade 1 (34.9%) or Grade 2 (41.0%). A total of 17 patients (20.5%) experienced a TEAE of Grade ≥3: 14 patients (16.9%) had Grade 3 TEAEs, 2 patients (2.4%) had Grade 4 TEAEs, and 1 patient (1.2%) had a Grade 5 TEAE.

### Treatment-Related TEAEs (TRAEs)

A total of 60 of 83 patients (72.3%) had at least 1 TEAE considered potentially related to Compound A in Part A (dose escalation).

The most common TRAEs, observed in ≥10% patients, included fatigue, nausea, arthralgia, constipation, hot flush and headache (Table 1). Most patients experienced TRAEs that were Grade 1 (41.0%) or Grade 2 (24.1%). Six patients (7.2%) experienced Grade 3 TRAEs: nausea (1 patient), headache (1 patient), aspartate aminotransferase increased (1 patient), electrocardiogram QT prolonged (2 patients), amylase increased (1 patient) and lipase increased (1 patient). There were no Grade 4 or Grade 5 TRAEs reported in Part A.

TABLE 12
TRAE by Preferred Term and Maximum CTCAE Grade
(Compound A, Part A) - All Dose Group-
All-Treated Analysis Set

| | Compound A - All Dose Group | | | | | |
|---|---|---|---|---|---|---|
| AE Preferred | Any Grade | Grade 1 | Grade 2 | Grade 3 | Grade 4 | Grade 5 |
| Term [n (%)] | (N = 83) | (N = 83) | (N = 83) | (N = 83) | (N = 83) | (N = 83) |
| Any treatment- | 60 (72.3) | 34 (41.0) | 20 (24.1) | 6 (7.2) | 0 | 0 |
| related AE | | | | | | |
| Fatigue | 22 (26.5) | 17 (20.5) | 5 (6.0) | 0 | 0 | 0 |
| Nausea | 21 (25.3) | 16 (19.3) | 4 (4.8) | 1 (1.2) | 0 | 0 |
| Arthralgia | 12 (14.5) | 11 (13.3) | 1 (1.2) | 0 | 0 | 0 |
| Constipation | 11 (13.3) | 10 (12.0) | 1 (1.2) | 0 | 0 | 0 |
| Hot flush | 11 (13.3) | 9 (10.8) | 2 (2.4) | 0 | 0 | 0 |
| Headache | 9 (10.8) | 8 (9.6) | 0 | 1 (1.2) | 0 | 0 |
| Decreased appetite | 8 (9.6) | 6 (7.2) | 2 (2.4) | 0 | 0 | 0 |
| Vomiting | 8 (9.6) | 7 (8.4) | 1 (1.2) | 0 | 0 | 0 |
| Aspartate | 7 (8.4) | 5 (6.0) | 1 (1.2) | 1 (1.2) | 0 | 0 |
| aminotransferase | | | | | | |
| increased | | | | | | |
| Diarrhea | 6 (7.2) | 6 (7.2) | 0 | 0 | 0 | 0 |
| Dry mouth | 6 (7.2) | 6 (7.2) | 0 | 0 | 0 | 0 |
| Electrocardiogram | 5 (6.0) | 2 (2.4) | 1 (1.2) | 2 (2.4) | 0 | 0 |
| QT prolonged | | | | | | |
| Hyperhidrosis | 4 (4.8) | 4 (4.8) | 0 | 0 | 0 | 0 |
| Alanine | 3 (3.6) | 3 (3.6) | 0 | 0 | 0 | 0 |
| aminotransferase | | | | | | |
| increased | | | | | | |
| Cough | 3 (3.6) | 3 (3.6) | 0 | 0 | 0 | 0 |
| Insomnia | 3 (3.6) | 3 (3.6) | 0 | 0 | 0 | 0 |
| Myalgia | 3 (3.6) | 3 (3.6) | 0 | 0 | 0 | 0 |
| Abdominal pain | 2 (2.4) | 2 (2.4) | 0 | 0 | 0 | 0 |
| Abdominal pain | 2 (2.4) | 1 (1.2) | 1 (1.2) | 0 | 0 | 0 |
| upper | | | | | | |
| Alopecia | 2 (2.4) | 2 (2.4) | 0 | 0 | 0 | 0 |
| Amylase increased | 2 (2.4) | 0 | 1 (1.2) | 1 (1.2) | 0 | 0 |
| Back pain | 2 (2.4) | 1 (1.2) | 1 (1.2) | 0 | 0 | 0 |
| Blood alkaline | 2 (2.4) | 2 (2.4) | 0 | 0 | 0 | 0 |
| phosphatase | | | | | | |
| increased | | | | | | |
| Blood creatine | 2 (2.4) | 1 (1.2) | 1 (1.2) | 0 | 0 | 0 |
| phosphokinase | | | | | | |
| increased | | | | | | |
| Bradycardia | 2 (2.4) | 2 (2.4) | 0 | 0 | 0 | 0 |
| Dry skin | 2 (2.4) | 2 (2.4) | 0 | 0 | 0 | 0 |
| Dyspepsia | 2 (2.4) | 1 (1.2) | 1 (1.2) | 0 | 0 | 0 |
| Hyperphosphatemia | 2 (2.4) | 2 (2.4) | 0 | 0 | 0 | 0 |
| Lipase increased | 2 (2.4) | 0 | 1 (1.2) | 1 (1.2) | 0 | 0 |
| Muscle spasms | 2 (2.4) | 2 (2.4) | 0 | 0 | 0 | 0 |
| Neutropenia* | 2 (2.4) | 0 | 2 (2.4) | 0 | 0 | 0 |
| Night sweats | 2 (2.4) | 2 (2.4) | 0 | 0 | 0 | 0 |
| Pain | 2 (2.4) | 2 (2.4) | 0 | 0 | 0 | 0 |
| Rash maculo- | 2 (2.4) | 1 (1.2) | 1 (1.2) | 0 | 0 | 0 |
| papular | | | | | | |
| Vision blurred | 2 (2.4) | 1 (1.2) | 1 (1.2) | 0 | 0 | 0 |
| Vulvovaginal | 2 (2.4) | 2 (2.4) | 0 | 0 | 0 | 0 |
| dryness | | | | | | |
| White blood cell | 2 (2.4) | 1 (1.2) | 1 (1.2) | 0 | 0 | 0 |
| count decreased | | | | | | |
| Abdominal | 1 (1.2) | 1 (1.2) | 0 | 0 | 0 | 0 |
| distension | | | | | | |
| Affect lability | 1 (1.2) | 1 (1.2) | 0 | 0 | 0 | 0 |
| Anemia | 1 (1.2) | 0 | 1 (1.2) | 0 | 0 | 0 |
| Asthenia | 1 (1.2) | 1 (1.2) | 0 | 0 | 0 | 0 |
| Blood lactate | 1 (1.2) | 1 (1.2) | 0 | 0 | 0 | 0 |
| dehydrogenase | | | | | | |
| increased | | | | | | |
| Bone pain | 1 (1.2) | 0 | 1 (1.2) | 0 | 0 | 0 |
| Carpal tunnel | 1 (1.2) | 0 | 1 (1.2) | 0 | 0 | 0 |
| syndrome | | | | | | |
| Chest discomfort | 1 (1.2) | 1 (1.2) | 0 | 0 | 0 | 0 |
| Chills | 1 (1.2) | 1 (1.2) | 0 | 0 | 0 | 0 |
| Dehydration | 1 (1.2) | 0 | 1 (1.2) | 0 | 0 | 0 |
| Dermatitis | 1 (1.2) | 1 (1.2) | 0 | 0 | 0 | 0 |
| acneiform | | | | | | |
| Diverticulitis | 1 (1.2) | 1 (1.2) | 0 | 0 | 0 | 0 |
| Dizziness | 1 (1.2) | 1 (1.2) | 0 | 0 | 0 | 0 |
| Dyspareunia | 1 (1.2) | 0 | 1 (1.2) | 0 | 0 | 0 |
| Dysphagia | 1 (1.2) | 0 | 1 (1.2) | 0 | 0 | 0 |
| Embolism venous | 1 (1.2) | 0 | 0 | 1 (1.2) | 0 | 0 |
| Flatulence | 1 (1.2) | 1 (1.2) | 0 | 0 | 0 | 0 |
| Gastroesophageal | 1 (1.2) | 0 | 1 (1.2) | 0 | 0 | 0 |
| reflux disease | | | | | | |
| Glossina | 1 (1.2) | 1 (1.2) | 0 | 0 | 0 | 0 |
| Hypercalcemia | 1 (1.2) | 1 (1.2) | 0 | 0 | 0 | 0 |
| Hyperglycemia | 1 (1.2) | 1 (1.2) | 0 | 0 | 0 | 0 |
| Hyperlipidemia | 1 (1.2) | 1 (1.2) | 0 | 0 | 0 | 0 |
| Hypermagnesemia | 1 (1.2) | 1 (1.2) | 0 | 0 | 0 | 0 |
| Hypertension | 1 (1.2) | 1 (1.2) | 0 | 0 | 0 | 0 |
| Hypokalemia | 1 (1.2) | 1 (1.2) | 0 | 0 | 0 | 0 |
| Malaise | 1 (1.2) | 1 (1.2) | 0 | 0 | 0 | 0 |
| Mouth ulceration | 1 (1.2) | 1 (1.2) | 0 | 0 | 0 | 0 |
| Muscular | 1 (1.2) | 1 (1.2) | 0 | 0 | 0 | 0 |
| weakness | | | | | | |
| Musculoskeletal | 1 (1.2) | 1 (1.2) | 0 | 0 | 0 | 0 |
| pain | | | | | | |
| Oedema peripheral | 1 (1.2) | 1 (1.2) | 0 | 0 | 0 | 0 |
| Pain in extremity | 1 (1.2) | 1 (1.2) | 0 | 0 | 0 | 0 |
| Painful respiration | 1 (1.2) | 1 (1.2) | 0 | 0 | 0 | 0 |
| Platelet count | 1 (1.2) | 0 | 1 (1.2) | 0 | 0 | 0 |
| decreased | | | | | | |
| Pollakiuria | 1 (1.2) | 1 (1.2) | 0 | 0 | 0 | 0 |
| Pruritus | 1 (1.2) | 1 (1.2) | 0 | 0 | 0 | 0 |
| Rash | 1 (1.2) | 0 | 1 (1.2) | 0 | 0 | 0 |
| Restlessness | 1 (1.2) | 1 (1.2) | 0 | 0 | 0 | 0 |
| Skin lesion | 1 (1.2) | 0 | 1 (1.2) | 0 | 0 | 0 |
| Stomatitis | 1 (1.2) | 1 (1.2) | 0 | 0 | 0 | 0 |

*Neutropenia was categorized according to the MedDRA preferred terms neutropenia and neutrophil count decreased.
Adverse Events were coded using MedDRA version 22.0.
Data Cutoff date: 6 Jun. 2023
TRAE ≥ 5% by Preferred Term and Maximum CTCAE Grade (Part A) - All Dose Group All-Treated Analysis Set

### Serious Adverse Events

A total of 11 (13.3%) SAEs were reported in Part A.

Grade 3 SAEs reported were pericardial effusion, dysphagia, stomatitis, lung infection, pneumonia staphylococcal, humerus fracture, procedural pain, electrocardiogram QT prolonged, embolism, embolism venous, malignant pleural effusion, spinal cord compression, and acute kidney injury 1 (1.2%) each.

Grade 4 SAE of electrocardiogram QT prolonged and Neutrophil count decreased 1 (1.2%) each were reported. One (1.2%) participant reported Grade 5 SAE in 500 mg dose group due to cardiac arrest assessed as not related to study drug.

### Deaths

Death was reported in a total of 33 (39.8%) of 83 participants in Part A. Two (2) of these deaths occurred within 30 days of last study treatment dose and were related to disease under study.

### TEAEs Leading to Discontinuation

Part A—Dose Escalation (n=83 patients): Five patients (6.0%) experienced a TEAE that led to discontinuation of Compound A in Part A.
One patient dosed at 100 mg QD experienced Grade 3 pericardial effusion deemed unrelated to Compound A.
One patient dosed at 100 mg QD experienced a Grade 3 pneumonia staphylococcal that was deemed unrelated to Compound A.
One patient dosed at 180 mg QD experienced Grade 1 ALT increased, Grade 1 GERD, Grade 3 blood ALP increased, and Grade 4 neutropenia, all of which were deemed unrelated to Compound A.
One patient dosed at 360 mg QD experienced a TRAE of Grade 3 venous embolism following a minor procedure (bone biopsy) that was deemed possibly related to Compound A by the investigator.
One patient dosed at 500 mg QD experienced Grade 2 fatigue, which was assessed as unrelated to Compound A.
   Part B of VERITAC—Dose Expansion (n=71 Patients)

### Treatment-Emergent AEs (TEAEs)

As of 6 Jun. 2023, a total of 63 of the 71 patients (88.7%) in Part B (dose expansion) reported at least 1 TEAE. The most commonly reported TEAEs (regardless of attribution to study drug) observed in ≥100% of patients, listed in decreasing order of frequency, include fatigue, nausea (26.8%), arthralgia (21.1%), constipation (19.7%), decreased appetite (15.5%), anemia (14.1%), hot flush (14.1%), aspartate aminotransferase increased (12.7%), headache (11.3%), and back pain (11.3%).

Most patients who experienced TEAEs reported Grade 1 (22.5%) or Grade 2 (33.8%) TEAEs. There were 30 TEAEs of Grade ≥3 severity: 20 patients (28.2%) had Grade 3 TEAEs, 2 patients (2.8%) had Grade 4 TEAEs, and 1 patient (1.4%) had a Grade 5 TEAE.

### Treatment-Related TEAEs (TRAEs)

A total of 54 patients (76.1%) of the 71 patients experienced at least 1 TEAE considered potentially related to Compound A (Table 13).

The most common TRAEs, observed in ≥10% of patients, include fatigue (36.6%), nausea (21.1%), arthralgia (14.1%) and hot flush (12.7%).

Most patients experienced TRAEs of Grade 1 (29.6%) or Grade 2 (38.0%). Five patients (7.0%) experienced 6 TRAEs with severity of Grade ≥3, including Grade 3 events of electrocardiogram QT prolonged 1 (1.4%), thrombocytopenia, neutropenia, decreased appetite, fatigue and accidental overdose (no AE was reported). There was 1 Grade 4 TRAE of hyperbilirubinemia. There were no Grade 5 TRAEs reported.

TABLE 1
TRAE by Preferred Term and Maximum CTCAE Grade
Compound A, Part B) - All Dose Group-
All-Treated Analysis Set

| | Compound A - All Dose Group | | | | | |
|---|---|---|---|---|---|---|
| | Any | Grade | Grade | Grade | Grade | Grade |
| AE Preferred | Grade | 1 | 2 | 3 | 4 | 5 |
| Term [n (%)] | (N = 71) | (N = 71) | (N = 71) | (N = 71) | (N = 71) | (N = 71) |
| Any treatment- | 54 (76.1) | 21 (29.6) | 27 (38.0) | 5 (7.0) | 1 (1.4) | 0 |
| related AE | | | | | | |
| Fatigue | 26 (36.6) | 16 (22.5) | 9 (12.7) | 1 (1.4) | 0 | 0 |
| Nausea | 15 (21.1) | 9 (12.7) | 6 (8.5) | 0 | 0 | 0 |
| Arthralgia | 10 (14.1) | 10 (14.1) | 0 | 0 | 0 | 0 |
| Hot flush | 9 (12.7) | 9 (12.7) | 0 | 0 | 0 | 0 |
| Aspartate | 7 (9.9) | 5 (7.0) | 2 (2.8) | 0 | 0 | 0 |
| aminotransferase | | | | | | |
| increased | | | | | | |
| Blood alkaline | 6 (8.5) | 5 (7.0) | 1 (1.4) | 0 | 0 | 0 |
| phosphatase | | | | | | |
| increased | | | | | | |
| Decreased appetite | 6 (8.5) | 2 (2.8) | 3 (4.2) | 1 (1.4) | 0 | 0 |
| Alanine | 5 (7.0) | 2 (2.8) | 3 (4.2) | 0 | 0 | 0 |
| aminotransferase | | | | | | |
| increased | | | | | | |
| Constipation | 5 (7.0) | 5 (7.0) | 0 | 0 | 0 | 0 |
| Insomnia | 5 (7.0) | 4 (5.6) | 1 (1.4) | 0 | 0 | 0 |
| Vomiting | 5 (7.0) | 3 (4.2) | 2 (2.8) | 0 | 0 | 0 |
| Electrocardiogram | 4 (5.6) | 1 (1.4) | 2 (2.8) | 1 (1.4) | 0 | 0 |
| QT prolonged | | | | | | |
| Neutropenia* | 4 (5.6) | 1 (1.4) | 2 (2.8) | 1 (1.4) | 0 | 0 |
| Anemia | 3 (4.2) | 1 (1.4) | 2 (2.8) | 0 | 0 | 0 |
| Headache | 3 (4.2) | 3 (4.2) | 0 | 0 | 0 | 0 |
| Vulvovaginal | 3 (4.2) | 3 (4.2) | 0 | 0 | 0 | 0 |
| dryness | | | | | | |
| White blood | 3 (4.2) | 2 (2.8) | 1 (1.4) | 0 | 0 | 0 |
| cell count | | | | | | |
| decreased | | | | | | |
| Abdominal | 2 (2.8) | 2 (2.8) | 0 | 0 | 0 | 0 |
| distension | | | | | | |
| Blood creatine | 2 (2.8) | 2 (2.8) | 0 | 0 | 0 | 0 |
| phosphokinase | | | | | | |
| increased | | | | | | |
| Chills | 2 (2.8) | 2 (2.8) | 0 | 0 | 0 | 0 |
| Diarrhea | 2 (2.8) | 2 (2.8) | 0 | 0 | 0 | 0 |
| Dry mouth | 2 (2.8) | 1 (1.4) | 1 (1.4) | 0 | 0 | 0 |
| Hyperglycemia | 2 (2.8) | 2 (2.8) | 0 | 0 | 0 | 0 |
| Myalgia | 2 (2.8) | 2 (2.8) | 0 | 0 | 0 | 0 |
| Neck pain | 2 (2.8) | 1 (1.4) | 1 (1.4) | 0 | 0 | 0 |
| Pain in extremity | 2 (2.8) | 2 (2.8) | 0 | 0 | 0 | 0 |
| Rash | 2 (2.8) | 2 (2.8) | 0 | 0 | 0 | 0 |
| Rash maculo- | 2 (2.8) | 2 (2.8) | 0 | 0 | 0 | 0 |
| papular | | | | | | |
| Abdominal pain | 1 (1.4) | 1 (1.4) | 0 | 0 | 0) | 0 |
| upper | | | | | | |
| Accidental | 1 (1.4) | 0 | 0 | 1 (1.4) | 0 | 0 |
| overdose | | | | | | |
| Activated partial | 1 (1.4) | 1 (1.4) | 0 | 0 | 0 | 0 |
| thromboplastin | | | | | | |
| time prolonged | | | | | | |
| Alopecia | 1 (1.4) | 1 (1.4) | 0 | 0 | 0 | 0 |
| Amylase increased | 1 (1.4) | 1 (1.4) | 0 | 0 | 0 | 0 |
| Anxiety | 1 (1.4) | 1 (1.4) | 0 | 0 | 0 | 0 |
| Blood bilirubin | 1 (1.4) | 1 (1.4) | 0 | 0 | 0 | 0 |
| increased | | | | | | |
| Blood lactate | 1 (1.4) | 1 (1.4) | 0 | 0 | 0 | 0 |
| dehydrogenase | | | | | | |
| increased | | | | | | |
| Bone pain | 1 (1.4) | 0 | 1 (1.4) | 0 | 0 | 0 |
| Bradycardia | 1 (1.4) | 1 (1.4) | 0 | 0 | 0 | 0 |
| Dehydration | 1 (1.4) | 0 | 1 (1.4) | 0 | 0 | 0 |
| Dry skin | 1 (1.4) | 1 (1.4) | 0 | 0 | 0 | 0 |
| Dysgeusia | 1 (1.4) | 1 (1.4) | 0 | 0 | 0 | 0 |
| Dyspepsia | 1 (1.4) | 0 | 1 (1.4) | 0 | 0 | 0 |
| Dyspnea exertional | 1 (1.4) | 1 (1.4) | 0 | 0 | 0 | 0 |
| Dysuria | 1 (1.4) | 1 (1.4) | 0 | 0 | 0 | 0 |
| Ear pain | 1 (1.4) | 1 (1.4) | 0 | 0 | 0 | 0 |
| Electrocardiogram | 1 (1.4) | 0 | 1 (1.4) | 0 | 0 | 0 |
| T wave abnormal | | | | | | |
| Eye hemorrhage | 1 (1.4) | 1 (1.4) | 0 | 0 | 0 | 0 |
| Face oedema | 1 (1.4) | 1 (1.4) | 0 | 0 | 0 | 0 |
| Feeling hot | 1 (1.4) | 1 (1.4) | 0 | 0 | 0 | 0 |
| Gastroesophageal | 1 (1.4) | 0 | 1 (1.4) | 0 | 0 | 0 |
| reflux disease | | | | | | |
| Hyperbilirubinemia | 1 (1.4) | 0 | 0 | 0 | 1 (1.4) | 0 |
| Hyperhidrosis | 1 (1.4) | 1 (1.4) | 0 | 0 | 0 | 0 |
| Hypertension | 1 (1.4) | 0 | 1 (1.4) | 0 | 0 | 0 |
| Hypokalemia | 1 (1.4) | 1 (1.4) | 0 | 0 | 0 | 0 |
| Hyponatremia | 1 (1.4) | 1 (1.4) | 0 | 0 | 0 | 0 |
| International | 1 (1.4) | 1 (1.4) | 0 | 0 | 0 | 0 |
| normalized ratio | | | | | | |
| increased | | | | | | |
| Irritability | 1 (1.4) | 1 (1.4) | 0 | 0 | 0 | 0 |
| Lymphocyte | 1 (1.4) | 0 | 1 (1.4) | 0 | 0 | 0 |
| count decreased | | | | | | |
| Muscular weakness | 1 (1.4) | 1 (1.4) | 0 | 0 | 0 | 0 |
| Musculoskeletal | 1 (1.4) | 0 | 1 (1.4) | 0 | 0 | 0 |
| chest pain | | | | | | |
| Musculoskeletal | 1 (1.4) | 1 (1.4) | 0 | 0 | 0 | 0 |
| pain | | | | | | |
| Musculoskeletal | 1 (1.4) | 1 (1.4) | 0 | 0 | 0 | 0 |
| stiffness | | | | | | |
| Oral dysesthesia | 1 (1.4) | 1 (1.4) | 0 | 0 | 0 | 0 |
| Palpitations | 1 (1.4) | 1 (1.4) | 0 | 0 | 0 | 0 |
| Paranesthesia | 1 (1.4) | 1 (1.4) | 0 | 0 | 0 | 0 |
| Polydipsia | 1 (1.4) | 1 (1.4) | 0 | 0 | 0 | 0 |
| Prothrombin | 1 (1.4) | 1 (1.4) | 0 | 0 | 0 | 0 |
| time prolonged | | | | | | |
| Pruritus | 1 (1.4) | 1 (1.4) | 0 | 0 | 0 | 0 |
| Rash erythematous | 1 (1.4) | 1 (1.4) | 0 | 0 | 0 | 0 |
| Thrombocytopenia | 1 (1.4) | 0 | 0 | 1 (1.4) | 0 | 0 |
| Uncoded | 1 (1.4) | 1 (1.4) | 0 | 0 | 0 | 0 |
| Urinary tract | 1 (1.4) | 0 | 1 (1.4) | 0 | 0 | 0 |
| infection | | | | | | |

*Neutropenia was categorized according to the MedDRA preferred terms neutropenia and neutrophil count decreased.
Adverse Events were coded using MedDRA version 22.0.
Data Cutoff date: 6 Jun. 2023
TRAE ≥ 5% by Preferred Term and Maximum CTCAE Grade (Part B) - All Dose Group All-Treated Analysis Set

### Serious Adverse Events

A total of 10 (14.1%) SAEs were reported in Part B.

Grade 3 SAEs were paranesthesia, seizure, spinal cord compression, intestinal obstruction, upper gastrointestinal hemorrhage, cholangitis, anaemia, thrombocytopenia, pyrexia and muscular weakness 1 (1.4%) each.

Grade 4 SAEs were hepatic hemorrhage, hyperbilirubinaemia, and hypercalcaemia 1 (1.4%) each. One (1.4%) participant reported Grade 5 SAE in 200 mg dose group due to acute respiratory failure(unrelated-due to underlying disease).

### Deaths

Death was reported in a total of 14 (19.7%) of 71 participants in Part B. Two (2) of these deaths occurred within 30 days of the last study treatment dose and were related to disease under study.

### TEAEs Leading to Discontinuation

Part B—Dose Expansion (n=71 patients): Four patients (5.6%) experienced a TEAE that led to discontinuation of Compound A in Part B:
One patient dosed at 200 mg QD experienced a TRAE of Grade 3 ECG QT prolonged.
One patient dosed at 200 mg QD experienced Grade 3 anemia deemed unrelated to Compound A.
One patient dosed at 500 mg QD experienced a TRAE of Grade 2 ECG T wave abnormal.
One patient experienced unrelated Grade 3 back pain and Grade 3 spinal cord compression.

### Part a and Part B Combined

Preliminary safety data from Part B (monotherapy dose expansion) is consistent with what has been observed in Part A (monotherapy dose escalation).

### Treatment-Emergent AEs (TEAEs)

The most common TEAEs observed in ≥10% of patients treated with Compound A (Monotherapy therapy Parts A and B) were fatigue (40.3%), nausea (29.9%), constipation (26.6%), arthralgia (22.1%), headache (16.2%), decreased appetite (15.6%), hot flush (15.6%), aspartate aminotransferase increased (13.6%), back pain (13.6%), vomiting (13.0%, diarrhea (12.3%), pain in extremity (11.0%), anemia (10.4%) and hyperglycaemia (10.4%) (Table 14).

TABLE 14
Treatment-emergent Adverse Events ≥10%
by Preferred Term (Part A + B)

| | Any Grade | Grade 3+ |
|---|---|---|
| AE Preferred Term [n (%)] | (N = 154) | (N = 154) |
| Any treatment-emergent AE | 143 (92.9) | 40 (26.0) |
| Fatigue | 62 (40.3) | 2 (1.3) |
| Nausea | 46 (29.9) | 1 (0.6) |
| Constipation | 41 (26.6) | 0 |
| Arthralgia | 34 (22.1) | 2 (1.3) |
| Headache | 25 (16.2) | 1 (0.6) |
| Decreased appetite | 24 (15.6) | 1 (0.6) |
| Hot flush | 24 (15.6) | 0 |
| Aspartate aminotransferase | 21 (13.6) | 3 (1.9) |
| increased | | |
| Back pain | 21 (13.6) | 2 (1.3) |
| Vomiting | 20 (13.0) | 0 |
| Diarrhea | 19 (12.3) | 0 |
| Pain in extremity | 17 (11.0) | 0 |
| Anemia | 16 (10.4) | 2 (1.3) |
| Hyperglycemia | 16 (10.4) | 0 |

Note:
Treatment-emergent is defined as AE occurring on/after date of first study dose and within 30 days of last study dose.
Note:
For each Preferred Term, patients are counted only once.
Note:
Adverse Events were coded using MedDRA version 22.0.

### Treatment-Related TEAEs (TRAEs)

The most common TRAEs observed in ≥10% of patients treated with Compound A (Monotherapy therapy Parts A and B) were fatigue (31.2%), nausea (23.4%), arthralgia (14.3%), hot flush (13.0%), and constipation (10.4%) (Table 15).

TABLE 15
Treatment-related Adverse Events by Preferred Term (Part A + B)

| | Any Grade | Grade 3+ |
|---|---|---|
| AE Preferred Term [n (%)] | (N = 154) | (N = 154) |
| Any treatment-related AE | 114 (74.0) | 12 (7.8) |
| Fatigue | 48 (31.2) | 1 (0.6) |
| Nausea | 36 (23.4) | 1 (0.6) |
| Arthralgia | 22 (14.3) | 0 |
| Hot flush | 20 (13.0) | 0 |
| Constipation | 16 (10.4) | 0 |

Note:
Treatment-emergent is defined as AE occurring on/after date of first study dose and within 30 days of last study dose.
Note:
Treatment-related AEs are those assessed as “Possibly Related”, “Probably Related” or “Related” by the Investigator.
Note:
For each Preferred Term, patients are counted only once.
Note:
Adverse Events were coded using MedDRA version 22.0.

### Phase 3 Monotherapy Study

An ongoing Phase 3, randomized, open-label, multicenter trial of Compound A vs fulvestrant in participants with ER+/HER2− advanced breast cancer whose disease progressed after prior endocrine-based treatment for advanced disease (VERITAC-2).

### All Causality Treatment-Emergent AE's

As of the data cutoff date, 6 patients were evaluable for AEs. The most common (≥20%) TEAE's were affective disorder, alanine aminotransferase increased, aspartate aminotransferase increased, COVID-19 [N=2 (33.3%) each].

### Treatment-Related AE's

Three (3) of 6 participants had a treatment-related AE. The most common treatment-related AE's (≥10%) were affective disorder [N=2 (33.3%)]; alanine aminotransferase increased, aspartate aminotransferase increased, hot flush, injection site pain, insomnia and pelvic pain [N=1 (16.7%) each].

### SAE

No SAEs were reported.

### Deaths

No deaths were reported.

### TEAEs Leading to Discontinuation

No discontinuations were reported due to TEAE.

### Phase 1 Monotherapy Studies

As of 6 Jun. 2023, 6 participants have been treated in a Phase 1 open-label single agent study in Japanese participants with ER+/HER2− locally advanced or metastatic breast cancer.

Nine (9) participants have been treated on a study in a Phase 1, open-label single agent study in Chinese participants with ER+/HER2− advanced breast cancer.

Participants in both these studies received Compound A 200 mg as a single agent.

TEAE's from participants in both studies have been combined and are described below. (Tables 16 and 17.)

### All Causality Treatment-Emergent AE's

Participants in both studies received Compound A 200 mg as a single agent. A total of 15 participants were evaluable for TEAE. Eleven (11) participants (73.3%) experienced a TEAE. Five (5) participants (33.4%) had grade 3-4 TEAE.

The most frequent all-causality TEAE (≥20%) were alanine aminotransferase increased [N=4 (26.7%)]; anemia [N=6 (40.0%)]; electrocardiogram QT prolonged [N=5 (33.3%)]; constipation, hyperuricemia, hypokalaemia, and neutrophil count decreased [N=3 (20.0%) each]. (Table 16.)

TABLE 16
Descending Order of Frequency of Treatment-Emergent Adverse Events by MedDRA
System Organ Class, Preferred Term and Maximum CTCAE Grade (All Causalities,
All Cycles) (Protocols C4891016 and C4891018)

| | Compound A 200 mg | | | | | | |
|---|---|---|---|---|---|---|---|
| Number of Participants | (N = 15) | | | | | | |
| Evaluable for AEs | | | | | | Missing | |
| Number (%) of Participants: | Grade | Grade | Grade | Grade | Grade | or | |
| by SYSTEM ORGAN CLASS | 1 | 2 | 3 | 4 | 5 | Unknown | Total |
| and Preferred Term | n (%) | n (%) | n (%) | n (%) | n (%) | n (%) | n (%) |
| Participants with events | 2 (13.3) | 4 (26.7) | 4 (26.7) | 1 (6.7) | 0 | 0 | 11 (73.3) |
| Investigations | 3 (20.0) | 1 (6.7) | 4 (26.7) | 0 | 0 | 0 | 8 (53.3) |
| Electrocardiogram QT | 3 (20.0) | 0 | 2 (13.3) | 0 | 0 | 0 | 5 (33.3) |
| prolonged | | | | | | | |
| Alanine aminotransferase | 4 (26.7) | 0 | 0 | 0 | 0 | 0 | 4 (26.7) |
| increased | | | | | | | |
| Neutrophil count decreased | 1 (6.7) | 1 (6.7) | 1 (6.7) | 0 | 0 | 0 | 3 (20.0) |
| Aspartate aminotransferase | 2 (13.3) | 0 | 0 | 0 | 0 | 0 | 2 (13.3) |
| increased | | | | | | | |
| White blood cell count | 1 (6.7) | 0 | 1 (6.7) | 0 | 0 | 0 | 2 (13.3) |
| decreased | | | | | | | |
| Bilirubin conjugated | 0 | 0 | 1 (6.7) | 0 | 0 | 0 | 1 (6.7) |
| increased | | | | | | | |
| Blood alkaline phosphatase | 1 (6.7) | 0 | 0 | 0 | 0 | 0 | 1 (6.7) |
| increased | | | | | | | |
| Blood bilirubin increased | 0 | 1 (6.7) | 0 | 0 | 0 | 0 | 1 (6.7) |
| Blood bilirubin | 1 (6.7) | 0 | 0 | 0 | 0 | 0 | 1 (6.7) |
| unconjugated increased | | | | | | | |
| Gamma-glutamyl | 1 (6.7) | 0 | 0 | 0 | 0 | 0 | 1 (6.7) |
| transferase increased | | | | | | | |
| Lymphocyte count | 1 (6.7) | 0 | 0 | 0 | 0 | 0 | 1 (6.7) |
| decreased | | | | | | | |
| Platelet count decreased | 1 (6.7) | 0 | 0 | 0 | 0 | 0 | 1 (6.7) |
| Blood and lymphatic system | 1 (6.7) | 4 (26.7) | 1 (6.7) | 0 | 0 | 0 | 6 (40.0) |
| disorders | | | | | | | |
| Anemia | 1 (6.7) | 4 (26.7) | 1 (6.7) | 0 | 0 | 0 | 6 (40.0) |
| Metabolism and nutrition | 4 (26.7) | 2 (13.3) | 0 | 0 | 0 | 0 | 6 (40.0) |
| disorders | | | | | | | |
| Hyperuricemia | 3 (20.0) | 0 | 0 | 0 | 0 | 0 | 3 (20.0) |
| Hypokalemia | 3 (20.0) | 0 | 0 | 0 | 0 | 0 | 3 (20.0) |
| Hypoalbuminemia | 1 (6.7) | 1 (6.7) | 0 | 0 | 0 | 0 | 2 (13.3) |
| Decreased appetite | 1 (6.7) | 0 | 0 | 0 | 0 | 0 | 1 (6.7) |
| Hypercalcemia | 0 | 1 (6.7) | 0 | 0 | 0 | 0 | 1 (6.7) |
| Hyperglycemia | 1 (6.7) | 0 | 0 | 0 | 0 | 0 | 1 (6.7) |
| Hyponatremia | 1 (6.7) | 0 | 0 | 0 | 0 | 0 | 1 (6.7) |
| Hypophosphatasemia | 1 (6.7) | 0 | 0 | 0 | 0 | 0 | 1 (6.7) |
| Gastrointestinal disorders | 5 (33.3) | 0 | 0 | 0 | 0 | 0 | 5 (33.3) |
| Constipation | 3 (20.0) | 0 | 0 | 0 | 0 | 0 | 3 (20.0) |
| Abdominal discomfort | 1 (6.7) | 0 | 0 | 0 | 0 | 0 | 1 (6.7) |
| Abdominal pain | 1 (6.7) | 0 | 0 | 0 | 0 | 0 | 1 (6.7) |
| Nausea | 1 (6.7) | 0 | 0 | 0 | 0 | 0 | 1 (6.7) |
| General disorders and | 0 | 3 (20.0) | 0 | 0 | 0 | 0 | 3 (20.0) |
| administration site conditions | | | | | | | |
| Pain | 0 | 2 (13.3) | 0 | 0 | 0 | 0 | 2 (13.3) |
| Chest discomfort | 0 | 1 (6.7) | 0 | 0 | 0 | 0 | 1 (6.7) |
| Fatigue | 1 (6.7) | 0 | 0 | 0 | 0 | 0 | 1 (6.7) |
| Pyrexia | 0 | 1 (6.7) | 0 | 0 | 0 | 0 | 1 (6.7) |
| Nervous system disorders | 2 (13.3) | 1 (6.7) | 0 | 0 | 0 | 0 | 3 (20.0) |
| Headache | 1 (6.7) | 1 (6.7) | 0 | 0 | 0 | 0 | 2 (13.3) |
| Dizziness | 1 (6.7) | 0 | 0 | 0 | 0 | 0 | 1 (6.7) |
| Skin and subcutaneous tissue | 3 (20.0) | 0 | 0 | 0 | 0 | 0 | 3 (20.0) |
| disorders | | | | | | | |
| Alopecia | 1 (6.7) | 0 | 0 | 0 | 0 | 0 | 1 (6.7) |
| Pruritus | 1 (6.7) | 0 | 0 | 0 | 0 | 0 | 1 (6.7) |
| Rash | 1 (6.7) | 0 | 0 | 0 | 0 | 0 | 1 (6.7) |
| Cardiac disorders | 1 (6.7) | 1 (6.7) | 0 | 0 | 0 | 0 | 2 (13.3) |
| Arteriosclerosis coronary | 0 | 1 (6.7) | 0 | 0 | 0 | 0 | 1 (6.7) |
| artery | | | | | | | |
| Sinus bradycardia | 1 (6.7) | 0 | 0 | 0 | 0 | 0 | 1 (6.7) |
| Immune system disorders | 0 | 1 (6.7) | 0 | 0 | 0 | 0 | 1 (6.7) |
| Seasonal allergy | 0 | 1 (6.7) | 0 | 0 | 0 | 0 | 1 (6.7) |
| Infections and infestations | 0 | 1 (6.7) | 0 | 0 | 0 | 0 | 1 (6.7) |
| COVID-19 | 0 | 1 (6.7) | 0 | 0 | 0 | 0 | 1 (6.7) |
| Injury, poisoning and | 1 (6.7) | 0 | 0 | 0 | 0 | 0 | 1 (6.7) |
| procedural complications | | | | | | | |
| Scar | 1 (6.7) | 0 | 0 | 0 | 0 | 0 | 1 (6.7) |
| Musculoskeletal and | 1 (6.7) | 0 | 0 | 0 | 0 | 0 | 1 (6.7) |
| connective tissue disorders | | | | | | | |
| Arthralgia | 1 (6.7) | 0 | 0 | 0 | 0 | 0 | 1 (6.7) |
| Neoplasms benign, malignant | 0 | 1 (6.7) | 0 | 0 | 0 | 0 | 1 (6.7) |
| and unspecified (incl cysts | | | | | | | |
| and polyps) | | | | | | | |
| Tumor pain | 0 | 1 (6.7) | 0 | 0 | 0 | 0 | 1 (6.7) |
| Renal and urinary disorders | 0 | 0 | 0 | 1 (6.7) | 0 | 0 | 1 (6.7) |
| Renal impairment | 0 | 0 | 0 | 1 (6.7) | 0 | 0 | 1 (6.7) |
| Respiratory, thoracic and | 1 (6.7) | 0 | 0 | 0 | 0 | 0 | 1 (6.7) |
| mediastinal disorders | | | | | | | |
| Painful respiration | 1 (6.7) | 0 | 0 | 0 | 0 | 0 | 1 (6.7) |

MedDRA v26.0 coding dictionary applied.
Cutoff date: 6 Jun. 2023

### Treatment-related TEAE

Eleven (11) of 15 participants had a TRAE. The most common TRAE (≥10%) were alanine aminotransferase increased and electrocardiogram QT prolonged [N=3 (20.0%) each]; Aspartate aminotransferase increased, constipation, hyperuricemia and neutrophil count decreased [N=2 (13.3%) each]. (Table 17.)

TABLE 17
Descending Order of Frequency of Treatment-Emergent Adverse Events by
MedDRA System Organ Class, Preferred Term, and Maximum CTCAE Grade
(Treatment Related, All Cycles)

| | Compound A 200 mg | | | | | | |
|---|---|---|---|---|---|---|---|
| Number of Participants | (N = 15) | | | | | | |
| Evaluable for AEs | | | | | | Missing | |
| Number (%) of Participants: by | Grade | Grade | Grade | Grade | Grade | or | |
| SYSTEM ORGAN CLASS | 1 | 2 | 3 | 4 | 5 | Unknown | Total |
| and Preferred Term | n (%) | n (%) | n (%) | n (%) | n (%) | n (%) | n (%) |
| Participants with events | 7 (46.7) | 2 (13.3) | 2 (13.3) | 0 | 0 | 0 | 11 (73.3) |
| Investigations | 4 (26.7) | 1 (6.7) | 2 (13.3) | 0 | 0 | 0 | 7 (46.7) |
| Alanine aminotransferase | 3 (20.0) | 0 | 0 | 0 | 0 | 0 | 3 (20.0) |
| increased | | | | | | | |
| Electrocardiogram QT | 2 (13.3) | 0 | 1 (6.7) | 0 | 0 | 0 | 3 (20.0) |
| prolonged | | | | | | | |
| Aspartate aminotransferase | 2 (13.3) | 0 | 0 | 0 | 0 | 0 | 2 (13.3) |
| increased | | | | | | | |
| Neutrophil count decreased | 1 (6.7) | 0 | 1 (6.7) | 0 | 0 | 0 | 2 (13.3) |
| Bilirubin conjugated increased | 0 | 1 (6.7) | 0 | 0 | 0 | 0 | 1 (6.7) |
| Blood bilirubin increased | 0 | 1 (6.7) | 0 | 0 | 0 | 0 | 1 (6.7) |
| Blood bilirubin unconjugated | 1 (6.7) | 0 | 0 | 0 | 0 | 0 | 1 (6.7) |
| increased | | | | | | | |
| Gamma-glutamyl transferase | 1 (6.7) | 0 | 0 | 0 | 0 | 0 | 1 (6.7) |
| increased | | | | | | | |
| Platelet count decreased | 1 (6.7) | 0 | 0 | 0 | 0) | 0 | 1 (6.7) |
| White blood cell count | 0 | 0 | 1 (6.7) | 0 | 0 | 0 | 1 (6.7) |
| decreased | | | | | | | |
| Gastrointestinal disorders | 4 (26.7) | 0 | 0 | 0 | 0 | 0 | 4 (26.7) |
| Constipation | 2 (13.3) | 0 | 0 | 0 | 0 | 0 | 2 (13.3) |
| Abdominal discomfort | 1 (6.7) | 0 | 0 | 0 | 0 | 0 | 1 (6.7) |
| Nausea | 1 (6.7) | 0 | 0 | 0 | 0 | 0 | 1 (6.7) |
| Metabolism and nutrition | 3 (20.0) | 0 | 0 | 0 | 0) | 0 | 3 (20.0) |
| disorders | | | | | | | |
| Hyperuricemia | 2 (13.3) | 0 | 0 | 0 | 0 | 0 | 2 (13.3) |
| Decreased appetite | 1 (6.7) | 0 | 0 | 0 | 0 | 0 | 1 (6.7) |
| Hypokalemia | 1 (6.7) | 0 | 0 | 0 | 0 | 0 | 1 (6.7) |
| Hypophosphatasemia | 1 (6.7) | 0 | 0 | 0 | 0 | 0 | 1 (6.7) |
| Nervous system disorders | 2 (13.3) | 0 | 0 | 0 | 0 | 0 | 2 (13.3) |
| Dizziness | 1 (6.7) | 0 | 0 | 0 | 0 | 0 | 1 (6.7) |
| Headache | 1 (6.7) | 0 | 0 | 0 | 0 | 0 | 1 (6.7) |
| Skin and subcutaneous tissue | 2 (13.3) | 0 | 0 | 0 | 0 | 0 | 2 (13.3) |
| disorders | | | | | | | |
| Alopecia | 1 (6.7) | 0 | 0 | 0 | 0 | 0 | 1 (6.7) |
| Pruritus | 1 (6.7) | 0 | 0 | 0) | 0 | 0 | 1 (6.7) |
| Blood and lymphatic system | 0 | 1 (6.7) | 0 | 0 | 0 | 0 | 1 (6.7) |
| disorders | | | | | | | |
| Anemia | 0 | 1 (6.7) | 0 | 0 | 0 | 0 | 1 (6.7) |
| Cardiac disorders | 1 (6.7) | 0 | 0 | 0 | 0 | 0 | 1 (6.7) |
| Sinus bradycardia | 1 (6.7) | 0 | 0 | 0 | 0 | 0 | 1 (6.7) |
| General disorders and | 0 | 1 (6.7) | 0 | 0) | 0 | 0 | 1 (6.7) |
| administration site conditions | | | | | | | |
| Fatigue | 1 (6.7) | 0 | 0 | 0 | 0 | 0 | 1 (6.7) |
| Pain | 0 | 1 (6.7) | 0 | 0 | 0 | 0 | 1 (6.7) |
| Musculoskeletal and connective | 1 (6.7) | 0) | 0 | 0 | 0 | 0 | 1 (6.7) |
| tissue disorders | | | | | | | |
| Arthralgia | 1 (6.7) | 0 | 0 | 0 | 0 | 0 | 1 (6.7) |

MedDRA v26.0 coding dictionary applied.
Cutoff date: 6 Jun. 2023

### SAE

As of the data cutoff date (6 Jun. 2023), 3 participants reported 3 SAEs. Bilirubin conjugated increased, and renal impairment was reported in study C4891018. Both were unrelated to Compound A.

### Deaths

No deaths were reported.

### TEAEs Leading to Discontinuation

No discontinuations reported due to TEAE.

Safety in Patients with Early Breast Cancer

A Phase 2, open-label, randomized, non-comparative proof of concept study of Compound A or anastrozole in participants with ER+/HER2− breast cancer amenable to definitive surgical resection is currently ongoing. The main goal of this study is to evaluate the biological activity of Compound A and anastrozole, respectively, by assessing the change in Ki-67 expression between baseline and 2 weeks post treatment with either Compound A or anastrozole (2:1 randomization, n=150). The study will also evaluate safety, tolerability, and clinical and pathological response after approximately 5.5 months of treatment.

As of 6 Jun. 2023, a total of 36 patients were evaluable for AEs (23 participants on Compound A 200 mg and 13 patients on anastrozole 1 mg). Thirteen of 36 (36.1%) patients had an adverse event.

### Treatment-Emergent AEs:

The most common (≥5%) treatment-emergent AEs were Hot flush (8.3%), Constipation (8.3%), Somnolence (5.6%), Hyperbilirubinemia (5.6%), hyperglycemia (5.6%) and Asthenia (5.6%). (Table 18.)

TABLE 18
Summary of Treatment-Emergent Adverse Events by MedDRA System
Organ Class, Preferred Term and Maximum CTCAE Grade (All Causalities,
All Cycles)

| Number of Participants | | | | | | |
|---|---|---|---|---|---|---|
| Evaluable for AEs | | | | | | |
| Number (%) of | | | | | | |
| Participants: by SYSTEM | Compound A 200 mg + Anastrozole 1 mg | | | | | |
| ORGAN CLASS and | Grade 1 | Grade 2 | Grade 3 | Grade 4 | Grade 5 | Total |
| Preferred Term | (N = 36) | (N = 36) | (N = 36) | (N = 36) | (N = 36) | (N = 36) |
| Participants with events | 12 (33.3) | 1 (2.8) | 0 | 0 | 0 | 13 (36.1) |
| Blood and lymphatic system | 2 (5.6) | 0 | 0 | 0 | 0 | 2 (5.6) |
| disorders | | | | | | |
| Anemia | 0 | 1 (2.8) | 0 | 0 | 0 | 1 (2.8) |
| Heparin-induced | 1 (2.8) | 0 | 0 | 0 | 0 | 1 (2.8) |
| thrombocytopenia | | | | | | |
| Lymphopenia | 1 (2.8) | 0 | 0 | 0 | 0 | 1 (2.8) |
| Cardiac disorders | 1 (2.8) | 0 | 0 | 0 | 0 | 1 (2.8) |
| Sinus bradycardia | 1 (2.8) | 0 | 0 | 0 | 0 | 1 (2.8) |
| Gastrointestinal disorders | 5 (13.9) | 0 | 0 | 0 | 0 | 5 (13.9) |
| Constipation | 3 (8.3) | 0 | 0 | 0 | 0 | 3 (8.3) |
| Diarrhea | 1 (2.8) | 0 | 0 | 0 | 0 | 1 (2.8) |
| Dyspepsia | 1 (2.8) | 0 | 0 | 0 | 0 | 1 (2.8) |
| Odynophagia | 1 (2.8) | 0 | 0 | 0 | 0 | 1 (2.8) |
| Stomatitis | 1 (2.8) | 0 | 0 | 0 | 0 | 1 (2.8) |
| General disorders and | 4 (11.1) | 0 | 0 | 0 | 0 | 4 (11.1) |
| administration site | | | | | | |
| conditions | | | | | | |
| Asthenia | 2 (5.6) | 0 | 0 | 0 | 0 | 2 (5.6) |
| Fatigue | 1 (2.8) | 0 | 0 | 0 | 0 | 1 (2.8) |
| Pyrexia | 1 (2.8) | 0 | 0 | 0 | 0 | 1 (2.8) |
| Hepatobiliary disorders | 3 (8.3) | 0 | 0 | 0 | 0 | 3 (8.3) |
| Hyperbilirubinemia | 2 (5.6) | 0 | 0 | 0 | 0 | 2 (5.6) |
| Hypertransaminasemia | 1 (2.8) | 0 | 0 | 0 | 0 | 1 (2.8) |
| Injury, poisoning and | 1 (2.8) | 0 | 0 | 0 | 0 | 1 (2.8) |
| procedural complications | | | | | | |
| Procedural pain | 1 (2.8) | 0 | 0 | 0 | 0 | 1 (2.8) |
| Investigations | 1 (2.8) | 1 (2.8) | 0 | 0 | 0 | 2 (5.6) |
| Blood uric acid increased | 1 (2.8) | 0 | 0 | 0 | 0 | 1 (2.8) |
| Hematocrit decreased | 0 | 1 (2.8) | 0 | 0 | 0 | 1 (2.8) |
| Metabolism and nutrition | 2 (5.6) | 0 | 0 | 0 | 0 | 2 (5.6) |
| disorders | | | | | | |
| Hypercalcemia | 1 (2.8) | 0 | 0 | 0 | 0 | 1 (2.8) |
| Hyperglycemia | 1 (2.8) | 1 (2.8) | 0 | 0 | 0 | 2 (5.6) |
| Hypernatremia | 1 (2.8) | 0 | 0 | 0 | 0 | 1 (2.8) |
| Hypokalemia | 1 (2.8) | 0 | 0 | 0 | 0 | 1 (2.8) |
| Hyponatremia | 1 (2.8) | 0 | 0 | 0 | 0 | 1 (2.8) |
| Musculoskeletal and | 1 (2.8) | 0 | 0 | 0 | 0 | 1 (2.8) |
| connective tissue disorders | | | | | | |
| Arthralgia | 1 (2.8) | 0 | 0 | 0 | 0 | 1 (2.8) |
| Myalgia | 1 (2.8) | 0 | 0 | 0 | 0 | 1 (2.8) |
| Nervous system disorders | 2 (5.6) | 0 | 0 | 0 | 0 | 2 (5.6) |
| Somnolence | 2 (5.6) | 0 | 0 | 0 | 0 | 2 (5.6) |
| Psychiatric disorders | 2 (5.6) | 0 | 0 | 0 | 0 | 2 (5.6) |
| Insomnia | 1 (2.8) | 0 | 0 | 0 | 0 | 1 (2.8) |
| Tension | 1 (2.8) | 0 | 0 | 0 | 0 | 1 (2.8) |
| Reproductive system and | 1 (2.8) | 0 | 0 | 0 | 0 | 1 (2.8) |
| breast disorders | | | | | | |
| Breast pain | 1 (2.8) | 0 | 0 | 0 | 0 | 1 (2.8) |
| Respiratory, thoracic and | 1 (2.8) | 0 | 0 | 0 | 0 | 1 (2.8) |
| mediastinal disorders | | | | | | |
| Asphyxia | 1 (2.8) | 0 | 0 | 0 | 0 | 1 (2.8) |
| Skin and subcutaneous tissue | 1 (2.8) | 0 | 0 | 0 | 0 | 1 (2.8) |
| disorders | | | | | | |
| Pruritus | 1 (2.8) | 0 | 0 | 0 | 0 | 1 (2.8) |
| Vascular disorders | 3 (8.3) | 0 | 0 | 0 | 0 | 3 (8.3) |

MedDRA v26.0 coding dictionary applied.
Cut-off date: 6 Jun. 2023

### Treatment-Emergent, Treatment Related AE's:

Seven participants (19.4%) on Compound A had a treatment-related AE. The most common (≥5%) treatment-related AEs were Asthenia (5.6%), Constipation (5.6%), Somnolence (5.6%) and Hot flush (5.6%). (Table 19.)

TABLE 19
Summary of Treatment-Emergent Adverse Events by MedDRA System Organ Class,
Preferred Term and Maximum CTCAE Grade (Treatment Related, All Cycles)

| Number of Participants Evaluable for AEs | Compound A | Anastrozole 1 mg | Total |
|---|---|---|---|
| Number (%) of Participants: by SYSTEM | 200 mg (N = 23) | (N = 13) | (N = 36) |
| ORGAN CLASS and Preferred Term | n (%) | n (%) | n (%) |
| With Any Adverse Event | 11 (47.8) | 2 (15.4) | 13 (36.1) |
| Blood and lymphatic system disorders | 1 (4.3) | 1 (7.7) | 2 (5.6) |
| Anemia | 0 | 1 (7.7) | 1 (2.8) |
| Heparin-induced thrombocytopenia | 1 (4.3) | 0 | 1 (2.8) |
| Lymphopenia | 0 | 1 (7.7) | 1 (2.8) |
| Cardiac disorders | 1 (4.3) | 0 | 1 (2.8) |
| Sinus bradycardia | 1 (4.3) | 0 | 1 (2.8) |
| Gastrointestinal disorders | 4 (17.4) | 1 (7.7) | 5 (13.9) |
| Constipation | 2 (8.7) | 1 (7.7) | 3 (8.3) |
| Diarrhea | 1 (4.3) | 0 | 1 (2.8) |
| Dyspepsia | 0 | 1 (7.7) | 1 (2.8) |
| Odynophagia | 1 (4.3) | 0 | 1 (2.8) |
| Stomatitis | 1 (4.3) | 0 | 1 (2.8) |
| General disorders and administration site | 4 (17.4) | 0 | 4 (11.1) |
| conditions | | | |
| Asthenia | 2 (8.7) | 0 | 2 (5.6) |
| Fatigue | 1 (4.3) | 0 | 1 (2.8) |
| Pyrexia | 1 (4.3) | 0 | 1 (2.8) |
| Hepatobiliary disorders | 3 (13.0) | 0 | 3 (8.3) |
| Hyperbilirubinemia | 2 (8.7) | 0 | 2 (5.6) |
| Hypertransaminasemia | 1 (4.3) | 0 | 1 (2.8) |
| Injury, poisoning and procedural complications | 1 (4.3) | 0 | 1 (2.8) |
| Procedural pain | 1 (4.3) | 0 | 1 (2.8) |
| Investigations | 1 (4.3) | 1 (7.7) | 2 (5.6) |
| Blood uric acid increased | 1 (4.3) | 0 | 1 (2.8) |
| Hematocrit decreased | 0 | 1 (7.7) | 1 (2.8) |
| Metabolism and nutrition disorders | 2 (8.7) | 0 | 2 (5.6) |
| Hypercalcemia | 1 (4.3) | 0 | 1 (2.8) |
| Hyperglycemia | 2 (8.7) | 0 | 2 (5.6) |
| Hypernatremia | 1 (4.3) | 0 | 1 (2.8) |
| Hypokalemia | 1 (4.3) | 0 | 1 (2.8) |
| Hyponatremia | 1 (4.3) | 0 | 1 (2.8) |
| Musculoskeletal and connective tissue disorders | 0 | 1 (7.7) | 1 (2.8) |
| Arthralgia | 0 | 1 (7.7) | 1 (2.8) |
| Myalgia | 0 | 1 (7.7) | 1 (2.8) |
| Nervous system disorders | 2 (8.7) | 0 | 2 (5.6) |
| Somnolence | 2 (8.7) | 0 | 2 (5.6) |
| Psychiatric disorders | 2 (8.7) | 0 | 2 (5.6) |
| Insomnia | 1 (4.3) | 0 | 1 (2.8) |
| Tension | 1 (4.3) | 0 | 1 (2.8) |
| Reproductive system and breast disorders | 1 (4.3) | 0 | 1 (2.8) |
| Breast pain | 1 (4.3) | 0 | 1 (2.8) |
| Respiratory, thoracic and mediastinal disorders | 1 (4.3) | 0 | 1 (2.8) |
| Asphyxia | 1 (4.3) | 0 | 1 (2.8) |
| Skin and subcutaneous tissue disorders | 1 (4.3) | 0 | 1 (2.8) |
| Pruritus | 1 (4.3) | 0 | 1 (2.8) |
| Vascular disorders | 3 (13.0) | 0 | 3 (8.3) |
| Hot flush | 3 (13.0) | 0 | 3 (8.3) |

MedDRA v26.0 coding dictionary applied.
Cut-off date: 6 JUN. 2023

### Serious Adverse Events

No SAEs were reported.

### Deaths

No deaths were reported.

### TEAEs Leading to Discontinuations

No study discontinuations reported due to TEAE.

### Example 11. In Vitro Studies

In a panel of ER+ cell lines, Compound A treatment resulted in dose-dependent decreases in ER levels, similar to those observed with fulvestrant. In MCF7 cells, Compound A achieved a DC₅₀ of 0.9 nM, with a maximum ER degradation of >90%. No degradation of ER was seen in MCF7 cells treated with Compound A and an excess of the cereblon ligand lenalidomide or with the proteasome inhibitor, carfilzomib (data not shown). Without being bound by theory, these observations support the proposed mode of action of Compound A in degrading ER via the proteasome by hijacking the intended intracellular E3 ligase.

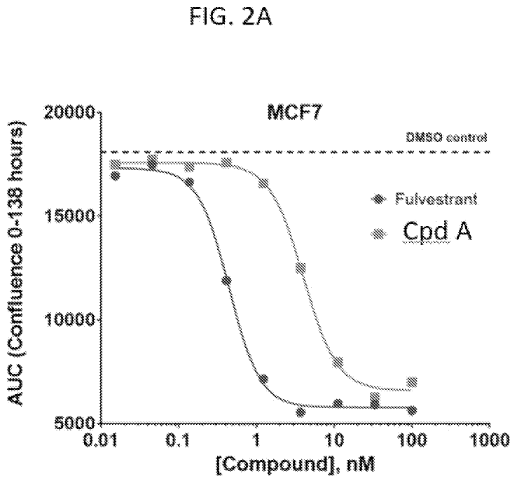

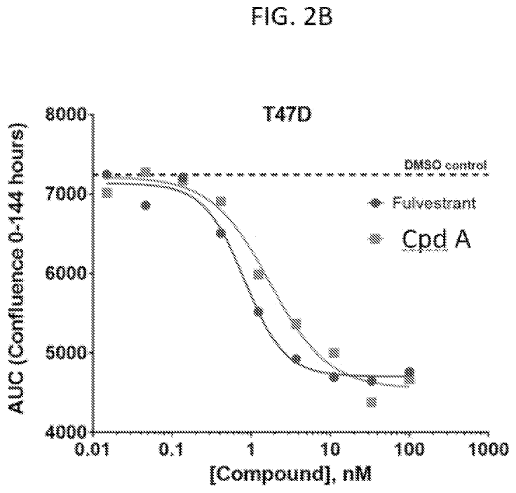

In functional studies characterizing the effects of Compound A mediated ER degradation Compound A caused a concentration-dependent decrease in proliferation of ER+ MCF7 and T47D cells with GI₅₀ values of 4 nM and 1.7 nM, respectively (FIGS. 2A and 2B).

### Example 12. In Vivo Studies

Compound A did not increase uterine wet weight in juvenile rats, demonstrating that it has no inherent ER agonist activity.

### Anti-Tumor Activity Following Single Agent Compound a Treatment in the MCF7 Xenograft Mouse Model

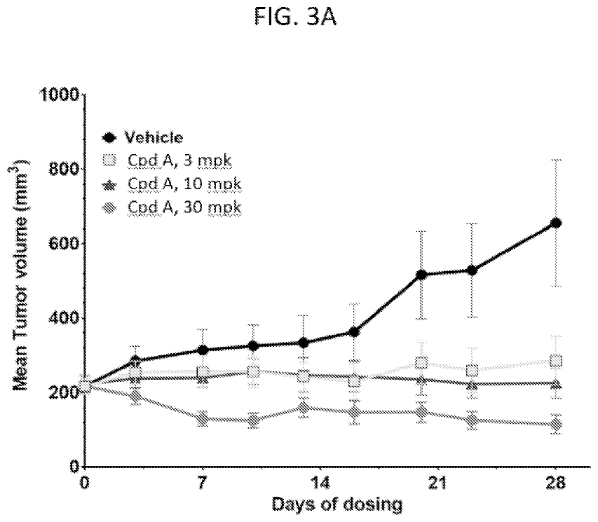

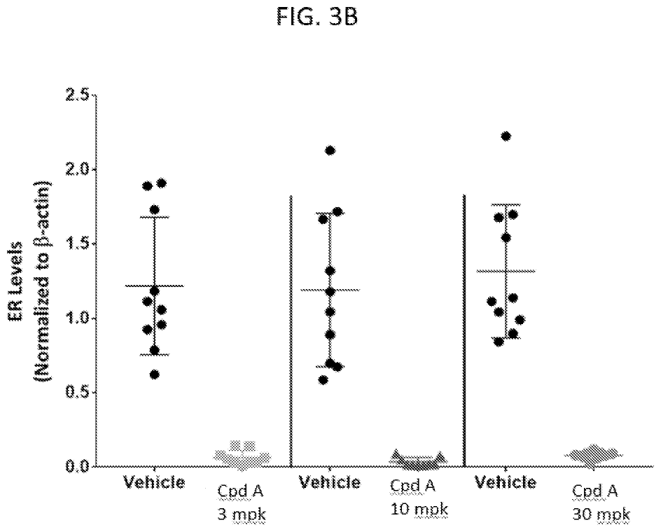

In a MCF7-xenograft mouse model, 3 to 30 mg/kg Compound A, orally administered to mice QD for 28 days, displayed dose-dependent efficacy (FIG. 3A) with doses of 3 and 10 mg/kg/day inhibiting tumor growth by 85% and 98%, respectively, relative to vehicle and 30 mg/kg/day leading to tumor shrinkage (124% TGI). The 30 mg/kg dose was associated with a mean AUC₀₋₂₄ of 5,717 ng·h/mL. At study termination, the tumor ER levels were reduced by ≥94% at all doses compared to mice administered vehicle only, suggesting that higher doses/exposures are required for maximal efficacy than for maximal ER degradation (FIG. 3B). Taken together, these data demonstrate that Compound A displays potent anti-tumor activity and robust ER degradation in a well-established ER+BC tumor model.

### Anti-Tumor Activity Following Single Agent Compound a Treatment in the Hormone-Independent ST941/HI PDX Mouse Model

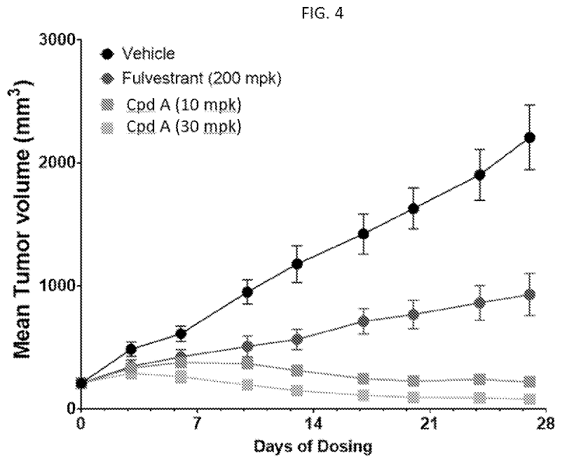

To explore in vivo activity of Compound A against a clinically relevant ER mutation associated with resistance to endocrine therapies, TGI studies were performed in the hormone-independent ST941/HI PDX model, which harbors a Y537S mutation in the ligand-binding domain. As shown in FIG. 4. Compound A at both 10 and 30 mg/kg/day demonstrated superior TGI (99% and 106% TGI, respectively) in the ST941/HI PDX model when compared to fulvestrant (62% TGI). Compound A (30 mg/kg) reduced ER by 88%, and fulvestrant reducing ER levels by 63% at study termination, despite fulvestrant achieving terminal plasma levels (250 nM) in mice treated with this dosing regimen and schedule that were approximately 5-fold above the reported clinical Cmax levels following IM administration of fulvestrant at 500 mg). These data demonstrate that Compound A is more active than a current standard of care agent, fulvestrant, in ER+BC models.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

The methods of the disclosure have been described herein by reference to certain preferred embodiments. However, as particular variations thereon will become apparent to those skilled in the art, based on the disclosure set forth herein, the disclosure is not to be considered as limited thereto.

Unless otherwise defined, all technical and scientific terms used herein have the same meaning as commonly understood by one of ordinary skill in the art to which this disclosure belongs. In the specification and claims, the singular forms also include the plural unless the context clearly dictates otherwise.

It is to be understood that at least some of the descriptions of the disclosure have been simplified to focus on elements that are relevant for a clear understanding of the disclosure, while eliminating, for purposes of clarity, other elements that those of ordinary skill in the art will appreciate may also comprise a portion of the disclosure. However, because such elements are well known in the art, and because they do not necessarily facilitate a better understanding of the disclosure, a description of such elements is not provided herein.

Further, to the extent that a method does not rely on the particular order of steps set forth herein, the particular order of the steps recited in a claim should not be construed as a limitation on that claim.

All patents, patent applications, references and publications cited herein are fully and completely incorporated by reference as if set forth in their entirety. Such documents are not admitted to be prior art to the present disclosure.

We claim:

1. A method for treating cancer comprising administering to a subject a daily dose of Compound A: 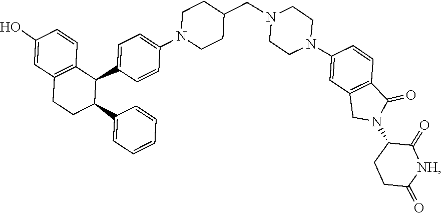 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]
or a pharmaceutically acceptable salt thereof, wherein the daily dose of Compound A is about 100 mg or about 200 mg.

2. A method for treating cancer comprising administering to a subject a daily dose of Compound A: 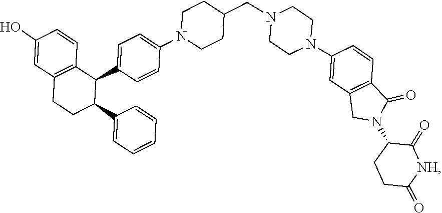 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]
wherein the daily dose of Compound A is about 200 mg.

3. A method for treating cancer comprising administering to a subject a daily dose of Compound A: 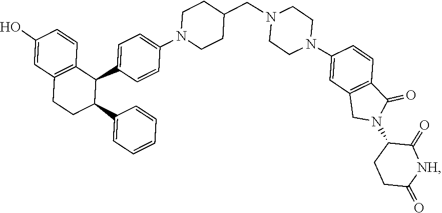 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]
wherein the daily dose of Compound A is about 100 mg.

4. The method of any one of claims 1 to 3, wherein the daily dose is administered once per day (QD).

5. The method of any one of claims 1 to 4, wherein the daily dose is administered orally to the subject.

6. The method of any one of claims 1 to 5, wherein the subject is in a fed state.

7. The method of any one of claims 1 to 6, wherein the cancer is breast cancer, lung cancer, colon cancer, brain cancer, head and neck cancer, prostate cancer, stomach cancer, pancreatic cancer, ovarian cancer, melanoma, endocrine cancer, uterine cancer, testicular cancer, or bladder cancer.

8. The method of claim 7, wherein the cancer is breast cancer, lung cancer, prostate cancer, pancreatic cancer, or ovarian cancer.

9. The method of claim 8, wherein the cancer is breast cancer, lung cancer, or prostate cancer.

10. The method of claim 9, wherein the cancer is breast cancer.

11. The method of claim 10, wherein the breast cancer is metastatic or locally advanced.

12. The method of claim 10 or 11, wherein the breast cancer is estrogen receptor positive (ER+) breast cancer.

13. The method of claim 12, wherein the estrogen receptor positive (ER+) breast cancer is human epidermal growth factor receptor 2 negative (HER2−).

14. The method of any one of claims 1 to 13, wherein the subject is human.
